(19) United States
(12) Patent Application Publication
(10) Pub. No.: US 2024/0076368 A1
(43) Pub. Date: Mar. 7, 2024

(54) METHODS OF CLASSIFYING AND TREATING PATIENTS

(71) Applicant: Scipher Medicine Corporation, Waltham, MA (US)

(72) Inventors: Viatcheslav R. AKMAEV, Sudbury, MA (US); Theodore R. MELLORS, Boston, MA (US)

(21) Appl. No.: 18/465,093

(22) Filed: Sep. 11, 2023

Related U.S. Application Data
(63) Continuation of application No. PCT/US2022/020815, filed on Mar. 17, 2022.
(60) Provisional application No. 63/163,414, filed on Mar. 19, 2021.
(60) Provisional application No. 63/306,054, filed on Feb. 2, 2022.

(51) Int. Cl. C07K 16/24; A61K 38/17; A61P 19/02; G16B 20/20; G16B 40/20; G16H 20/10; G16H 50/20

(52) U.S. Cl. CPC C07K 16/241; A61K 38/1793; A61P 19/02; G16B 20/20; G16B 40/20; G16H 20/10; G16H 50/20

(57) ABSTRACT

Presented herein are systems and methods for developing classifiers useful for predicting response to particular treatments. For example, in some embodiments, the present disclosure provides methods of treating subjects suffering from an autoimmune disorder, the method comprising: administering an anti-TNF therapy to subjects who have been determined to be responsive via a classifier established to distinguish between responsive and non-responsive prior subjects in a cohort who have received the anti-TNF therapy. For example, in some embodiments, the present disclosure provides methods of treating subjects suffering from an autoimmune disorder during therapeutic treatment, the method comprising: identifying responsive and non-responsive prior subjects over a time period beginning from the administering of the anti-TNF therapy.

### CROSS-REFERENCE TO RELATED APPLICATIONS

The present application is a continuation of International Application No. PCT/US2022/020815, filed Mar. 17, 2022, which claims priority to U.S. Provisional App. No. 63/163,414, filed Mar. 19, 2021, and U.S. Provisional App. No. 63/306,054, filed Feb. 2, 2022, each of which is incorporated by reference herein in its entirety.

### BACKGROUND

Autoimmune diseases such as rheumatoid arthritis (RA) affect millions of patients, and their treatments represent a significant component of overall healthcare expenditure. Autoimmune diseases can be divided into two groups—organ-specific and systemic autoimmunity. Rheumatoid diseases including RA belong to the systemic autoimmune diseases which primarily manifests in synovial joints and eventually causes irreversible destruction of tendons, cartilage, and bone. Although there is no current cure for RA, significant improvements have been made to manage the treatment of these patients mainly through the development of anti-TNF (tumor necrosis factor) agents, which act to neutralize the pro-inflammatory signaling of this cytokine. Such biologic therapies (e.g., Humira®, Enbrel, Remicade®, Simponi®, and Cimzia®) have significantly improved the treatment outcome of some RA patients.

Roughly 34% of RA patients (a low percentage) show a clinical response to anti-TNF therapies, achieving low disease activity (LDA) and sometimes achieving remission. Disease progression in these so called “responder” patients, is likely a result of inappropriate TNF-driven pro-inflammatory responses. For patients failing to respond to anti-TNFs, there are alternative approved therapies available such as anti-CD20, co-stimulation blockade, JAK and anti-IL6 therapy. However, patients may be switched onto such alternative therapy after first cycling through different anti-TNFs, which can take over a year, while symptoms persist and the disease progresses further, making it more difficult to reach treatment targets. In addition to the problem of delay in treatment, risks of serious infection and malignancy associated with anti-TNF therapy are so significant that product approvals may require so-called “black box warnings” be included on the label. Other potential side effects of such therapy include, for example, congestive heart failure, demyelinating disease, and other systemic side effects.

### SUMMARY

A significant problem with anti-TNF therapies is that response rates are inconsistent. Regardless of the measure used to define response, a subset of RA patients may an adequate response to TNFi treatment: 50-70% achieve ACR20, 30-40% achieve ACR50, and 15-25% achieve ACR70 response and 10-25% achieve remission. Many studies have attempted to identify biomarkers and develop models to predict response to TNFi therapy before the initiation of treatment. Failure to validate and reproduce the performance of these predictive biomarkers in new patient populations and clinical trials was a typical outcome. Differing characteristics between patient populations, laboratory methods and procedures in generating molecular data and other biases inherent to single-cohort retrospective blood studies have hindered precision medicine progress not only in rheumatology but in other medical specialties as well.

In some aspects, the methods and compositions described herein permit care providers to distinguish between or among categories of subjects—e.g., subjects likely to benefit from a particular therapy (e.g., anti-TNF therapy) from those who are not, those who are more likely to achieve or suffer a particular outcome or side effect, etc. In some embodiments, such provided technologies thus reduce risks to patients, increase timing and quality of care for non-responder patient populations, increase efficiency of drug development, or avoid costs associated with administering ineffective therapy to non-responder patients or with treating side effects such patients experience upon receiving the relevant therapy (e.g., anti-TNF therapy).

In some aspects, the present disclosure provides methods of treating subjects with particular therapy (e.g., anti-TNF therapy), in some embodiments, a method comprising: administering a therapy to subjects who have been determined to be responsive via a classifier established to distinguish between subjects expected to be responsive vs non-responsive to the therapy. In some embodiments, a classifier identifies 60% or greater of non-responders within a treatment-naive cohort. In some embodiments, a classifier identifies 60% or greater of non-responders within a treatment-naive cohort of at least 350 subjects.

A classifier can be a molecular signature response classifier derived from differences in gene expression between known responders and non-responders within a cohort. In some embodiments, one or more genes having statistically significant differences in expression between responders and non-responders are included as part of a molecular signature response classifier. In some embodiments, proteins associated with genes having statistically significant differences in expression between responders and non-responders are mapped onto a human interactome to validate relationship between selected genes and disease biology.

Provided classifiers further incorporate additional elements, e.g., clinical characteristics or single nucleotide polymorphisms useful for classifying response or non-response in a given patient.

In some embodiments, the present disclosure provides methods of treating subjects suffering from an autoimmune disorder, in some embodiments, a method comprising: administering an anti-TNF therapy to subjects who have been determined to be responsive via a classifier established to distinguish between responsive and non-responsive prior subjects in a cohort who have received the anti-TNF therapy; wherein a classifier is developed by assessing: one or more genes whose expression levels significantly correlate (e.g., in a linear or non-linear manner) to clinical responsiveness or non-responsiveness; at least one of: presence of one or more single nucleotide polymorphisms (SNPs) in an expressed sequence of the one or more genes; or at least one clinical characteristic of the responsive and non-responsive prior subjects; and wherein the classifier is validated by an independent cohort than the cohort who have received the anti-TNF therapy.

In some embodiments, the subject has been previously administered the anti-TNF therapy. In some embodiments, the subject has been administered the anti-TNF therapy at least one, at least two, at least three, at least four, at least five, or at least six months prior to said administering.

In some embodiments, a classifier identifies 60% or greater of non-responders within a treatment-naive cohort. In some embodiments, a classifier identifies 60% or greater of non-responders within a treatment-naive cohort of at least 350 subjects.

In some embodiments, one or more genes are characterized by their topological properties when mapped on a human interactome map. In some embodiments, SNPs are identified in reference to a human genome. In some embodiments, a classifier is developed by assessing each of: the one or more genes whose expression levels significantly correlate (e.g., in a linear or non-linearmanner) toclinicalresponsivenessornon-responsiveness; presenceoftheoneormore SNPs; and the at least one clinical characteristic.

In some embodiments, one or more genes comprise: ALPL, ATRAID, BCL6, CDK11A, CFLAR, COMMD5, GOLGA1, IL1B, IMPDH2, JAK3, KLHDC3, LIMK2, NOD2, NOTCH1, SPINT2, SPON2, STOML2, TRIM25, or ZFP36.

In some embodiments, one or more genes comprise: ALPL, BCL6, CDK11A, CFLAR, IL1B, JAK3, LIMK2, NOD2, NOTCH1, TRIM25, or ZFP36.

In some embodiments, at least one clinical characteristic is selected from: body-mass index (BMI), gender, age, race, previous therapy treatment, disease duration, C-reactive protein level, presence of anti-cyclic citrullinated peptide, presence of rheumatoid factor, patient global assessment, treatment response rate (e.g., ACR20, ACR50, ACR70), and combinations thereof.

In some embodiments, anti-TNF therapy comprises administration of infliximab, adalimumab, etanercept, cirtolizumab pegol, golilumab, or biosimilars thereof. In some embodiments, a disease, disorder, or condition is selected from rheumatoid arthritis, psoriatic arthritis, ankylosing spondylitis, Crohn's disease, ulcerative colitis, chronic psoriasis, hidradenitis suppurativa, multiple sclerosis, and juvenile idiopathic arthritis. In some embodiments, a classifier is established using microarray analysis derived from responsive and non-responsive prior subjects. In some embodiments, a classifier is validated using RNAseq data derived from the independent cohort. In some embodiments, the SNPs are selected from Table 3.

In some embodiments, the present disclosure provides a system for classifying a subject suffering from an autoimmune disease as likely responsive or likely non-responsive to an anti-TNF therapy prior to any administration of said anti-TNF therapy to said subject, the system comprising: a processor; and a memory having instructions thereon, the instructions, when executed by the processor, causing the processor to: (a) receive a set of data, said set of data comprising an expression level for the subject of each of one or more genes comprising: ALPL, ATRAID, BCL6, CDK11A, CFLAR, COMMD5, GOLGA1, IL1B, IMPDH2, JAK3, KLHDC3, LIMK2, NOD2, NOTCH1, SPINT2, SPON2, STOML2, TRIM25, or ZFP36.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede or take precedence over any such contradictory material.

### BRIEF DESCRIPTION OF THE DRAWINGS

FIG. 1 is an example embodiment of proteins encoded by transcripts predictive of response were mapped onto the human interactome. Proteins are shown in circles and pair-wise physical protein-protein interactions are indicated as lines. The RA disease module is composed of seed genes (red) and DIAMOnD genes (teal). The proteins encoded by eleven transcript features (squares) were significantly connected to the RA disease module (p-value <0.05).

FIGS. 2A-2D illustrate cross-validation of the molecular signature response classifier (“MSRC”) among 245 patients from the Corrona CERTAIN study. FIG. 2A illustrates a receiver operator curve for stratification of patients based on CDAI, DAS28-CRP, ACR70 and ACR50 clinical outcomes. FIG. 2B illustrates a comparison of model scores for patients with or without a molecular signature of non-response. Boxes and intersecting line depict interquartile range and median, respectively. Bisecting colored lines indicate change in mean. Ratio of the percentage of patients with or without a molecular signature of non-response in CDAI remission, LDA, moderate or high disease activity illustrated in FIG. 2C (CDAI) and FIG. 2D (DAS28-CRP). Bars indicate a greater proportion of patients with a molecular signature when above 1.0 or without a molecular signature when below 1.0. NA: not applicable, no patients in category.

FIGS. 3A-3F illustrate validation of the MSRC to identify patients naive to targeted therapies who are unlikely to respond to TNFi therapy. Receiver operator curve for stratification of patients based on CDAI, DAS28-CRP, ACR70 and ACR50 clinical outcomes at 3 months (FIG. 3A) and 6 months (FIG. 3B). Comparison of model scores at 3 months (FIG. 3C) and 6 months (FIG. 3D) for patients with or without a molecular signature of non-response. Boxes and intersecting line depict interquartile range and median, respectively. Bisecting colored lines indicate change in mean. Ratio of the percentage of patients with or without a molecular signature of non-response in CDAI remission, LDA, moderate or high disease activity per CDAI (FIG. 3E) and DAS28-CRP (FIG. 3F). Bars indicate a greater proportion of patients with a molecular signature when above 1.0 or without a molecular signature when below 1.0. NA: not applicable, no patients in category; NS: not significant.

FIGS. 4A-4B illustrate validation of the MSRC to identify TNFi-exposed patients who are unlikely to respond to TNFi therapy. FIG. 4A illustrates receiver operator curve for stratification of patients who are receiving a TNFi therapy based on achievement of CDAI remission or DAS28-CRP remission 3 months after test results. FIG. 4B illustrates comparison of model scores for patients with or without a molecular signature of non-response. Boxes and intersecting line depict interquartile range and median, respectively. Bisecting colored lines indicate change in mean.

FIG. 5 illustrates biology of inadequate response to TNFi therapies. The MSRC includes transcript that encode proteins involved in many aspects of RA pathophysiology: innate immune response, cytokine biosynthesis, T and B cell homeostasis, bone homeostasis, the unfolded protein response, autophagy, apoptosis and pro-inflammatory signaling.

FIG. 6 is a flow chart of study design. A subset of 345 patients from the CERTAIN study were analyzed: 100 for identification of transcript biomarkers of non-response to TNFi therapies and 245 for cross-validation. 273 patients enrolled in the NETWORK-004 prospective observational study; 244 passed initial enrollment screening, 194 completed the 3-month follow-up visit and 168 completed the 6-month follow-up visit. 87% (146/168) of patients who completed the study had complete molecular and clinical data required to perform validation analyses.

FIG. 7 is a Venn diagram showing breakdown of patients who provided samples at 3-months, 6-months, and at both 3-months and 6-months exposure to TNF therapy.

FIGS. 8A-8D provide ROC curves showing PrismRA performance among patient samples collected 3 months and 6 months after TNF initiation. FIG. 8A shows 3-month samples using +3-month outcome. FIG. 8B shows 3-month samples using +6-month outcome. FIG. 8C shows 6-month samples using +3-month outcome. FIG. 8D shows 6-month samples using +6-month outcome.

FIGS. 9A-9B provide ROC curves showing model performance among 122 patients that provide both 3-month and 6-month samples. FIG. 9A shows 3-month samples using +6-month endpoints. FIG. 9B shows 6-month samples using +3-month endpoints.

FIG. 10 provides an example computer system for executing methods according to some aspects or embodiments of the disclosure.

### DETAILED DESCRIPTION

A significant problem with various therapies (e.g., anti-TNF) therapies is that response rates are inconsistent. Indeed, recent international conferences designed to bring together leading scientists and clinicians in the fields of immunology and rheumatology to identify unmet needs in these fields almost universally identify uncertainty in response rates as an ongoing challenge. For example, the 19^{th} annual International Targeted Therapies meeting, which held break-out sessions relating to challenges in treatment of a variety of diseases, including rheumatoid arthritis, psoriatic arthritis, axial spondyloarthritis, systemic lupus erythematous, and connective tissue diseases (e.g, Sjogren's syndrome, systemic sclerosis, vasculitis including Bechet's and IgG4 related disease), identified certain issues common to all of these diseases, specifically, “the need for better understanding the heterogeneity within each disease . . . so that predictive tools for therapeutic response scan be developed. See Winthrop, et al.,” The unmet need in rheumatology: Reports from the targeted therapies meeting 2017,” Clin. Immunol. pii: S1521-6616(17)30543-0, Aug. 12, 2017, which is incorporated herein by reference for all purposes. Similarly, extensive literature relating to treatment of Crohn's Disease with anti-TNF therapy consistently bemoans erratic response rates and inability to predict which patients will benefit. See, e.g., M. T. Abreu, “Anti-TNF Failures in Crohn's Disease,” Gastroenterol Hepatol (NY), 7(1):37-39 (January 2011); see also Ding et al., “Systematic review: predicting and optimising response to anti-TNF therapy in Crohn's disease algorithm for practical management,” Aliment Pharmacol. Ther., 43(1):30-51 (January 2016), which is incorporated herein by reference for all purposes, (reporting that “[p]rimary nonresponse to anti-TNF treatment affects 13-40% of patients.”).

Thus, a significant number of patients to whom anti-TNF therapy is currently being administered do not benefit from the treatment, and can even be harmed. Risks of serious infection and malignancy associated with anti-TNF therapy are so significant that product approvals may require so-called “black box warnings” be included on the label. Other potential side effects of such therapy include, for example, congestive heart failure, demyelinating disease, and other systemic side effects. Furthermore, given that several weeks to months of treatment are required before a patient is identified as not responding to anti-TNF therapy (e.g., is a non-responder to anti-TNF therapy), proper treatment of such patients can be significantly delayed as a result of the current inability to identify responder vs non-responder subjects. See, e.g., Roda et al., “Loss of Response to Anti-TNFs: Definition, Epidemiology, and Management,” Clin. Trani. Gastroenterol., 7 (1):e035 (January 2016), which is incorporated herein by reference for all purposes, (citing Hanauer et al.,” ACCENT I Study group. Maintenance Infliximab for Crohn's disease: the ACCENT I randomized trial,” Lancet 59:1541-1549 (2002); Sands et al., “Infliximab maintenance therapy for fistulizing Crohn's disease,” N. Engl. J. Med. 350:876-885 (20004)).

Accordingly, in some embodiments, the present disclosure provides methods of treating subjects with anti-TNF therapy, the method comprising: administering the anti-TNF therapy to subjects who have been determined to be responsive via a classifier established to distinguish between responsive and non-responsive prior subjects who have received the anti-TNF therapy wherein the classifier that is developed by assessing: one or more genes whose expression levels significantly correlate (e.g., in a linear or non-linear manner) to clinical responsiveness or non-responsiveness; and at least one of: presence of one or more single nucleotide polymorphisms (SNPs) in an expressed sequence of the one or more genes; or at least one clinical characteristic of the responsive and non-responsive prior subjects.

Presented herein are systems and methods for the automated prediction of subject response to anti-TNF therapies. Also presented herein are modular systems for automated interpretation of genomic or multi-omic data.

As used herein, the term “administration” generally refers to the administration of a composition to a subject or system, for example to achieve delivery of an agent that is, or is included in or otherwise delivered by, the composition.

As used herein, the term “agent” generally refers to an entity (e.g., for example, a lipid, metal, nucleic acid, polypeptide, polysaccharide, small molecule, etc., or complex, combination, mixture or system [e.g., cell, tissue, organism] thereof), or phenomenon (e.g., heat, electric current or field, magnetic force or field, etc.).

As used herein, the term “amino acid” generally refers to any compound or substance that can be incorporated into a polypeptide chain, e.g., through formation of one or more peptide bonds. In some embodiments, an amino acid has the general structure H2N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. As used herein, the term “standard amino acid” refers to any of the twenty L-amino acids commonly found in naturally occurring peptides. “Nonstandard amino acid” refers to any amino acid, other than the standard aminoacids, regardless of whether itis or can be found in a natural source. In some embodiments, an amino acid, including a carboxy- or amino-terminal amino acid in a polypeptide, can contain a structural modification as compared to the general structure above. For example, in some embodiments, an amino acid may be modified by methylation, amidation, acetylation, pegylation, glycosylation, phosphorylation, or substitution (e.g., of the amino group, the carboxylic acid group, one or more protons, or the hydroxyl group) as compared to the general structure. In some embodiments, such modification may, for example, alter the stability or the circulating half-life of a polypeptide containing the modified amino acid as compared to one containing an otherwise identical unmodified amino acid. In some embodiments, such modification does not significantly alter a relevant activity of a polypeptide containing the modified amino acid, as compared to one containing an otherwise identical unmodified amino acid. As will be clear from context, in some embodiments, the term “amino acid” may be used to refer to a free amino acid; in some embodiments it may be used to refer to an amino acid residue of a polypeptide, e.g., an amino acid residue within a polypeptide. As used herein, the term “analog” generally refers to a substance that shares one or more particular structural features, elements, components, or moieties with a reference substance. Generally, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

As used herein, the term “antagonist” generally may refer to an agent, or condition whose presence, level, degree, type, or form is associated with a decreased level or activity of a target. An antagonist may include an agent of any chemical class including, for example, small molecules, polypeptides, nucleic acids, carbohydrates, lipids, metals, or any other entity that shows the relevant inhibitory activity. In some embodiments, an antagonist may be a “direct antagonist” in that it binds directly to its target; in some embodiments, an antagonist may be an “indirect antagonist” in that it exerts its influence by mechanisms other than binding directly to its target; e.g., by interacting with a regulator of the target, so that the level or activity of the target is altered). In some embodiments, an “antagonist” may be referred to as an “inhibitor”.

As used herein, the term “antibody” generally refers to a polypeptide that includes canonical immunoglobulin sequence elements sufficient to confer specific binding to a particular target antigen. Intact antibodies as produced in nature are, in some embodiments, approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. In some embodiments, each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). In some embodiments, a short region, referred to as the “switch”, connects the heavy chain variable and constant regions. The “hinge” connects CH2 and CH3 domains to the rest of the antibody. In some embodiments, two disulfide bonds in this hinge region connect the two heavy chain polypeptides to one another in an intact antibody. In some embodiments, each light chain is comprised of two domains—an amino-terminal variable (VL) domain, followed by a carboxy-terminal constant (CL) domain, separated from one another by another “switch”. In some embodiments, intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. In some embodiments, naturally-produced antibodies are also glycosylated, such as on the CH2 domain. Each domain in a natural antibody has, in some embodiments, a structure characterized by an “immunoglobulin fold” formed from two beta sheets (e.g., 3-, 4-, or 5-stranded sheets) packed against each other in a compressed antiparallel beta barrel. Each variable domain contains, in some embodiments, three hypervariable loops referred to as “complement determining regions” (CDR1, CDR2, and CDR3) and four somewhat invariant “framework” regions (FR1, FR2, FR3, and FR4). In some embodiments, when natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. In some embodiments, the Fc region of naturally-occurring antibodies binds to elements of the complement system, and also to receptors on effector cells, including for example effector cells that mediate cytotoxicity. In some embodiments, affinity or other binding attributes of Fc regions for Fc receptors can be modulated through glycosylation or other modification. In some embodiments, antibodies produced or utilized in accordance with the present disclosure include glycosylated Fc domains, including Fc domains with modified or engineered such glycosylation. For purposes of the present disclosure, in certain embodiments, any polypeptide or complex of polypeptides that includes sufficient immunoglobulin domain sequences as found in natural antibodies can be referred to or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc. Moreover, the term “antibody” as used herein, can refer in appropriate embodiments (unless otherwise stated or clear from context) to any constructs or formats for utilizing antibody structural and functional features in alternative presentation. For example, in some embodiments, an antibody utilized in accordance with the present disclosure is in a format selected from, but not limited to, intact IgA, IgG, IgE or IgM antibodies; bi- or multi-specific antibodies (e.g., Zybodies®, etc.); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VI-11-1s; Anticalins®; Nanobodies® minibodies; BiTE®s; ankyrin repeat proteins or DARPINs®; Avimers®; DARTs; TCR-like antibodies; Adnectins®; Affilins®; Trans-Bodies®; Affibodies®; TrimerX®; MicroProteins; Fynomers®, Centyrins®; and KALBITOR®. In some embodiments, an antibody may lack a covalent modification (e.g., attachment of a glycan) that it can have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]).

Two events or entities are “associated” generally with one another, as that term is used herein, if the presence, level, degree, type or form of one is correlated with that of the other. For example, a particular entity (e.g., polypeptide, genetic signature, metabolite, microbe, etc) is considered to be associated with a particular disease, disorder, or condition, if its presence, level or form correlates with incidence of or susceptibility to the disease, disorder, or condition (e.g., across a relevant population). In some embodiments, two or more entities are physically “associated” with one another if they interact, directly or indirectly, so that they are or remain in physical proximity with one another. In some embodiments, two or more entities that are physically associated with one another are covalently linked to one another; in some embodiments, two or more entities that are physically associated with one another are not covalently linked to one another but are non-covalently associated, for example by mechanisms of hydrogen bonds, van der Waals interaction, hydrophobic interactions, magnetism, and combinations thereof.

As used herein, the term “biological sample” generally refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, or excretions; or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate method. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of or by adding one or more agents to) a primary sample. For example, filtering using a semi-permeable membrane. Such a“processed sample” may comprise, for example nucleic acids or proteins extracted from a sample or obtained by subjecting a primary sample to techniques such as amplification or reverse transcription of mRNA, isolation or purification of certain components, etc.

As used herein, the term “combination therapy” generally refers to a clinical intervention in which a subject is simultaneously exposed to two or more therapeutic regimens (e.g., two or more therapeutic agents). In some embodiments, the two or more therapeutic regimens may be administered simultaneously. In some embodiments, the two or more therapeutic regimens may be administered sequentially (e.g., a first regimen administered prior to administration of any doses of a second regimen). In some embodiments, the two or more therapeutic regimens are administered in overlapping dosing regimens. In some embodiments, administration of combination therapy may involve administration of one or more therapeutic agents or modalities to a subject receiving the other agent(s) or modality. In some embodiments, combination therapy does not necessarily require that individual agents be administered together in a single composition (or even necessarily at the same time). In some embodiments, two or more therapeutic agents or modalities of a combination therapy are administered to a subject separately, e.g., in separate compositions, via separate administration routes (e.g., one agent orally and another agent intravenously), or at different time points. In some embodiments, two or more therapeutic agents may be administered together in a combination composition, or even in a combination compound (e.g., as part of a single chemical complex or covalent entity), via the same administration route, or at the same time.

As used herein, the term “comparable” generally refers to two or more agents, entities, situations, sets of conditions, etc., that may not be identical to one another but that are sufficiently similar to permit comparison there between so that conclusions may reasonably be drawn based on differences or similarities observed. In some embodiments, comparable sets of conditions, circumstances, individuals, or populations are characterized by a plurality of substantially identical features and one or a small number of varied features. It may be understood, in context, what degree of identity is required in any given circumstance for two or more such agents, entities, situations, sets of conditions, etc. to be considered comparable. For example, sets of circumstances, individuals, or populations may be comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

As used herein, the phrase “corresponding to” generally refers to a relationship between two entities, events, or phenomena that share sufficient features to be reasonably comparable such that “corresponding” attributes are apparent. For example, in some embodiments, the term may be used in reference to a compound or composition, to designate the position or identity of a structural element in the compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, may not actually be the 190^{th} amino acid in a particular amino acid chain but rather corresponds to the residue found at 190 in the reference polypeptide; various approaches may be used to identify “corresponding” amino acids. For example, there are various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides or nucleic acids in accordance with the present disclosure.

As used herein, the term “dosing regimen” generally refers to a set of unit doses (e.g., more than one) that are administered individually to a subject, e.g., separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (e.g., is a therapeutic dosing regimen).

As used herein, the terms “improved,” “increased,” or “reduced,” or grammatically comparable comparative terms thereof, generally indicate values that are relative to a comparable reference measurement. For example, in some embodiments, an assessed value achieved with an agent of interest may be “improved” relative to that obtained with a comparable reference agent. Alternatively or additionally, in some embodiments, an assessed value achieved in a subject or system of interest may be “improved” relative to that obtained in the same subject or system under different conditions (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc.).

As used herein, the term “pharmaceutical composition” generally refers to an active agent, formulated together with one or more pharmaceutically acceptable carriers. In some embodiments, the active agent is present in unit dose amounts appropriate for administration in a therapeutic regimen to a relevant subject (e.g., in amounts that have been demonstrated to show a statistically significant probability of achieving a predetermined therapeutic effect when administered), or in a different, comparable subject (e.g., in a comparable subject or system that differs from the subject or system of interest in presence of one or more indicators of a particular disease, disorder or condition of interest, or in prior exposure to a condition or agent, etc.). In some embodiments, comparative terms refer to statistically relevant differences (e.g., that are of a prevalence or magnitude sufficient to achieve statistical relevance).

As used herein, the phrase “pharmaceutically acceptable” generally refers to those compounds, materials, compositions, or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “reference” generally describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested or determined substantially simultaneously with the testing or determination of interest. In some embodiments, a reference or control is a historical reference or control, optionally embodied in a tangible medium. In some embodiments, a reference or control is determined or characterized under comparable conditions or circumstances to those under assessment. It may be determined when sufficient similarities are present to justify reliance on or comparison to a particular possible reference or control.

As used herein, the term “therapeutically effective amount” generally refers to an amount of a substance (e.g., a therapeutic agent, composition, or formulation) that elicits an intended biological response when administered as part of a therapeutic regimen. In some embodiments, a therapeutically effective amount of a substance is an amount that is sufficient, when administered to a subject suffering from or susceptible to a disease, disorder, or condition, to treat, diagnose, prevent, or delay the onset of the disease, disorder, or condition. As will be appreciated by those of ordinary skill in this art, the effective amount of a substance may vary depending on such factors as the intended biological endpoint, the substance to be delivered, the target cell or tissue, etc. For example, the effective amount of compound in a formulation to treat a disease, disorder, or condition is the amount that alleviates, ameliorates, relieves, inhibits, prevents, delays onset of, reduces severity of or reduces incidence of one or more symptoms or features of the disease, disorder or condition. In some embodiments, a therapeutically effective amount is administered in a single dose; in some embodiments, multiple unit doses are required to deliver a therapeutically effective amount.

As used herein, the term “variant” generally refers to an entity that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. Any biological or chemical reference entity has certain characteristic structural elements. A variant, by definition, is a distinct chemical entity that shares one or more such characteristic structural elements. To give but a few examples, a small molecule may have a characteristic core structural element (e.g., a macrocycle core) or one or more characteristic pendent moieties so that a variant of the small molecule is one that shares the core structural element and the characteristic pendent moieties but differs in other pendent moieties or in types of bonds present (single vs double, E vs Z, etc.) within the core, a polypeptide may have a characteristic sequence element comprised of a plurality of amino acids having designated positions relative to one another in linear or three-dimensional space or contributing to a particular biological function, a nucleic acid may have a characteristic sequence element comprised of a plurality of nucleotide residues having designated positions relative to on another in linear or three-dimensional space. For example, a variant polypeptide may differ from a reference polypeptide as a result of one or more differences in amino acid sequence or one or more differences in chemical moieties (e.g., carbohydrates, lipids, etc.) covalently attached to the polypeptide backbone. In some embodiments, a variant polypeptide shows an overall sequence identity with a reference polypeptide that is at least 85%, 86%, 87%, 88%, 89%, 90%, 91%, 92%, 93%, 94%, 95%, 96%, 97%, or 99%. Alternatively or additionally, in some embodiments, a variant polypeptide does not share at least one characteristic sequence element with a reference polypeptide. In some embodiments, the reference polypeptide has one or more biological activities. In some embodiments, a variant polypeptide shares one or more of the biological activities of the reference polypeptide. In some embodiments, a variant polypeptide lacks one or more of the biological activities of the reference polypeptide. In some embodiments, a variant polypeptide shows a reduced level of one or more biological activities as compared with the reference polypeptide. In many embodiments, a polypeptide of interest is considered to be a “variant” of a parent or reference polypeptide if the polypeptide of interest has an amino acid sequence that is identical to that of the parent but for a small number of sequence alterations at particular positions. For example, fewer than 20%, 15%, 10%, 9%, 8%, 7%, 6%, 5%, 4%, 3%, 2% of the residues in the variant are substituted as compared with the parent. In some embodiments, a variant has 10, 9, 8, 7, 6, 5, 4, 3, 2, or 1 substituted residue as compared with a parent. Often, a variant has a very small number (e.g., fewer than 5, 4, 3, 2, or 1) number of substituted functional residues (e.g., residues that participate in a particular biological activity). Furthermore, a variant may have not more than 5, 4, 3, 2, or 1 additions or deletions, and often has no additions or deletions, as compared with the parent. Moreover, any additions or deletions may be fewer than about 25, about 20, about 19, about 18, about 17, about 16, about 15, about 14, about 13, about 10, about 9, about 8, about 7, about 6, and commonly are fewer than about 5, about 4, about 3, or about 2 residues. In some embodiments, the parent or reference polypeptide is one found in nature.

A. Provided Classifier(s)

The present disclosure provides a classifier and development of such a classifier that can identify (e.g., predict) which patients will or will not respond to a particular therapy. In some embodiments, a classifier is established to distinguish between responsive and non-responsive prior subjects who have received an anti-TNF therapy (e.g., a particular anti-TNF agent or regimen).

Among other things, the present disclosure encompasses an insight that expression level(s) for a certain set of genes, alone and in combination with one another, optionally coupled with certain clinical characteristics or with presence or absence of certain single nucleotide polymorphism(s), are useful for predicting response (e.g., one or more features of response) to anti-TNF therapy.

In some embodiments, the present disclosure provides a classifier that is or includes such gene expression level(s), clinical characteristic(s) or SNP(s), and demonstrates that it has been established to distinguish between subjects who do and who do not respond to anti-TNF therapy. In some embodiments, a provided classifier is established to distinguish, through retrospective analysis of historical (e.g., prior) subject population(s) who received anti-TNF therapy and whose responsiveness is known (e.g., was previously determined), between subjects (e.g., anti-TNF therapy naive subjects) who are responsive or non-responsive to anti-TNF therapy. In some embodiments, a classifier that, when applied to such historical (e.g., prior) population(s) identifies at least 50% of non-responders within a cohort with at least 70% accuracy is considered “validated.” In some embodiments, a classifier that, when applied to such historical (e.g., prior) population(s) identifies at least 60% of non-responders within a cohort with at least 70% accuracy is considered “validated.” In some embodiments, a classifier that, when applied to such historical (e.g., prior) population(s) identifies at least 70% of non-responders within a cohort with at least 70% accuracy is considered “validated.” In some embodiments, a classifier that, when applied to such historical (e.g., prior) population(s) identifies at least 80% of non-responders within a cohort with at least 70% accuracy is considered “validated.” In some embodiments, a classifier that, when applied to such historical (e.g., prior) population(s) identifies at least 90% of non-responders within a cohort with at least 70% accuracy is considered “validated.” In some embodiments, a classifier that, when applied to such historical (e.g., prior) population(s) identifies at least 99% of non-responders within a cohort with at least 70% accuracy is considered “validated.”

In some embodiments, a classifier that, when applied to such historical (e.g., prior) population(s) identifies at least 50% of non-responders within a cohort with at least 80% accuracy is considered “validated.” In some embodiments, a classifier that, when applied to such historical (e.g., prior) population(s) identifies at least 50% of non-responders within a cohort with at least 90% accuracy is considered “validated.” In some embodiments, a classifier that, when applied to such historical (e.g., prior) population(s) identifies at least 50% of non-responders within a cohort with at least 99% accuracy is considered “validated.”

In some embodiments, the present disclosure provides methods of treating subjects suffering from a disease, disorder, or condition, comprising administering an anti-TNF therapy to a subject(s) that has been determined through application of a provided classifier to be likely to respond to such anti-TNF therapy; alternatively or additionally, in some embodiments, the present disclosure provides methods of treating subjects suffering from a disease, disorder or condition, comprising withholding anti-TNF therapy, or administering an alternative to anti-TNF therapy to a subject(s) determined through application of a provided classifier to be unlikely to respond to such anti-TNF therapy.

In some embodiments, a provided classifier may be or comprise gene expression information for one or more genes. Alternatively or additionally, in some embodiments, a provided classifier may be or comprise presence or absence of one or more single nucleotide polymorphisms (SNP) or one or more clinical features or characteristics of a relevant subject.

In some embodiments, a classifier is developed by assessing each of the one or more genes whose expression levels significantly correlate (e.g., in a linear or non-linear manner) to clinical responsiveness or non-responsiveness; presence of the one or more SNPs; and at least one clinical characteristic.

In some embodiments, as described herein, a classifier is developed by retrospective analysis of one or more features (e.g., gene expression levels, presence or absence of one or more SNPs, etc.) of biological samples from patients (e.g., prior subjects) who have received anti-TNF therapy and have been determined to respond (e.g., are responders) or not to respond (e.g., are non-responders); alternatively or additionally, in some embodiments, a classifier is developed by retrospective analysis of one or more clinical characteristics of such patients, which may or may not involve assessment of any biological samples (and may be accomplished, for example, by reference to medical records). In some embodiments, all such patients have received the same anti-TNF therapy (optionally for the same or different periods of time); alternatively or additionally, in some embodiments, all such patients have been diagnosed with the same disease, disorder or condition. In some embodiments, patients whose biological samples are analyzed in the retrospective analysis had received different anti-TNF therapy (e.g., with a different anti-TNF agent or according to a different regimen); alternatively or additionally, in some embodiments, patients whose biological samples are analyzed in the retrospective analysis have been diagnosed with different diseases, disorders, or conditions.

Many statistical classification techniques are suitable as approaches to perform the classification described above (e.g., distinguish between subjects who do and who do not respond to anti-TNF therapy). Such methods include but are not limited to supervised learning approaches.

In supervised learning approaches, a group of samples from two or more groups (e.g., those do and do not respond to anti-TNF therapy) are analyzed or processed with a statistical classification method. Absence/presence of genes or particular SNPs or variants, or expression level of genes or biomarkers described herein can be used as a basis for classifier that differentiates between the two or more groups. A new sample can then be analyzed or processed so that the classifier can associate the new sample with one of the two or more groups.

Commonly used supervised classifiers include without limitation the neural network (e.g, artificial neural network, multi-layer perceptron), support vector machines, k-nearest neighbours, Gaussian mixture model, Gaussian, naive Bayes, decision tree and radial basis function (RBF) classifiers. Linear classification methods include Fisher's linear discriminant, logistic regression, naive Bayes classifier, perceptron, and support vector machines (SVMs). Other classifiers for use with methods according to the disclosure include quadratic classifiers, k-nearest neighbor, boosting, decision trees, random forests, neural networks, pattern recognition, Bayesian networks and Hidden Markov models. Other classifiers, including improvements or combinations thereof, commonly used for supervised learning, can also be suitable for use with the methods described herein.

Classification using supervised methods can generally be performed by the following methodology:

1. Gather a training set. These can include, for example, expression levels of one or more genes or biomarkers described herein from a sample from a patient responding or not responding to anti-TNF therapy. The training samples are used to “train” the classifier.

2. Determine the input“feature” representation of the learned function. The accuracy of the learned function depends on how the input object is represented. For example, the input object is transformed into a feature vector, which contains a number of features that are descriptive of the object. The features might include a set of genes detected in a sample from a patient or subject.

3. Determine the structure of the learned function and corresponding learning algorithm. A learning algorithm is chosen, e.g., artificial neural networks, decision trees, Bayes classifiers or support vector machines. The learning algorithm is used to build the classifier.

4. Build the classifier (e.g., classification model). The learning algorithm is run on the gathered training set. Parameters of the learning algorithm may be adjusted by optimizing performance on a subset (called a validation set) of the training set, or via cross-validation. After parameter adjustment and learning, the performance of the algorithm may be measured on a test set of naive samples that is separate from the training set. The built model can involve feature coefficients or importance measures assigned to individual features.

In some cases, the individual features are individual genes or levels of individual genes. In some cases, the level of the gene is a normalized value, an average value, a median value, a mean value, an adjusted average, or other adjusted level or value. The individual features may comprise or consist of sets or panels of genes, such as the sets provided herein.

Once the classifier (e.g., classification model) is determined as described above (“trained”), it can be used to classify a sample, e.g., a patient sample comprising expressed genes that is analyzed or processed according to methods described herein.

1. Gene Expression

In some embodiments, a gene expression aspect of a classifier as described herein is determined by assessing one or more genes whose expression levels significantly correlate (e.g, in a linear or non-linear manner) to clinical responsiveness or non-responsiveness; and at least one of: presence of one or more single nucleotide polymorphisms (SNPs) in an expressed sequence of the one or more genes; or at least one clinical characteristic of the responsive and non-responsive prior subjects. Genes whose expression levels show statistically significant differences between the responder and non-responder populations may be included in the gene response signature.

In some embodiments, the present disclosure embodies an insight that the source of a problem with certain prior efforts to identify or provide a classifier between responsive and non-responsive subjects is through comparison of gene expression levels in responder vs non-responder populations have emphasized or focused on (often solely on) genes that show the largest difference (e.g., greater than 2-fold change) in expression levels between the populations. The present disclosure appreciates that even genes those expression level differences are relatively small (e.g, less than 2-fold change in expression) provide useful information and are valuably included in a classifier in embodiments described herein.

Moreover, in some embodiments, the present disclosure embodies an insight that analysis of interaction patterns of genes whose expression levels show statistically significant differences (optionally including small differences) between responder and non-responder populations as described herein provides new and valuable information that materially improves the quality and predictive power of a classifier.

In some embodiments a provided classifier is or comprises a gene or set of genes that can be used to determine (e.g., whose expression level correlates with) whether a subject will or will not respond to a particular therapy (e.g., anti-TNF therapy). In some embodiments, a classifier is developed by assessing one or more genes whose expression levels significantly correlate (e.g., in a linear or non-linear manner) to clinical responsiveness or non-responsiveness; and at least one of: presence of one or more single nucleotide polymorphisms (SNPs); and at least one clinical characteristic of the responsive and non-responsive prior subjects.

In some embodiments, one or more genes for use in a classifier and/or for measuring gene expression are selected from genes in Table 1, and combinations thereof:

TABLE 1

ALPL
ATRAID
BCL6
CDK11A
CFLAR
COMMD5
GOLGA1
IL1B
IMPDH2
JAK3
KLHDC3
LIMK2
NOD2
NOTCH1
SPINT2
SPON2
STOML2
TRIM25
ZFP36

In some embodiments, genes for use in a classifier or for measuring gene expression are selected from two or more genes from Table 1. In some embodiments, genes for use in a classifier or for measuring gene expression are selected from two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more or all nineteen genes from Table 1.

In some embodiments, genes for use in a classifier or for measuring gene expression are selected from one or more genes from Table 2, and combinations thereof:

TABLE 2

ALPL
BCL6
CDK11A
CFLAR
IL1B
JAK3
LIMK2
NOD2
NOTCH1
TRIM25
ZFP36

In some embodiments, genes for use in a classifier or for measuring gene expression are selected from two or more genes from Table 2. In some embodiments, genes for use in a classifier or for measuring gene expression are selected from two or more, three or more, four or more, five or more, six or more, seven or more, eight or more, nine or more, ten or more or all eleven genes from Table 2.

In some embodiments, a gene expression pattern in a classifier can be identified or detected using mRNA or protein expression datasets, for example as may be or have been prepared from validated biological data (e.g., biological data derived from publicly available databases such as Gene Expression Omnibus (“GEO”)). In some embodiments, a classifier may be derived by comparing gene expression levels of known responsive and known non-responsive prior subjects to a specific therapy (e.g., anti-TNF therapy). In some embodiments, certain genes (e.g., signature genes) are selected from this cohort of gene expression data to be used in developing the classifier.

In some embodiments, signature genes or expression patterns are identified by methods analogous to those reported by Santolini, “A personalized, multiomics approach identifies genes involved in cardiac hypertrophy and heart failure,” Systems Biology and Applications, (2018)4:12; doi:10.1038/s41540-018-0046-3, which is incorporated herein by reference for all purposes. In some embodiments, signature genes or expression patterns are identified by comparing gene expression levels of known responsive and non-responsive prior subjects and identifying significant changes between the two groups, wherein the significant changes can be large differences in expression (e.g., greater than 2-fold change), small differences in expression (e.g, less than 2-fold change), or both. In some embodiments, genes are ranked by significance of difference in expression. In some embodiments, significance is measured by Pearson correlation between gene expression and response outcome. In some embodiments, signature genes are selected from the ranking by significance of difference in expression. In some embodiments, the number of signature genes selected is less than the total number of genes analyzed. In some embodiments, 200 signature genes or less are selected. In some embodiments 100 genes or less are selected.

In some embodiments, signature genes are selected in conjunction with or are characterized by their location on a human interactome (HI), a map of protein-protein interactions. Use of the HI in this way encompasses a recognition that mRNA activity is dynamic and determines the actual over and under expression of proteins critical to understanding certain diseases. In some embodiments, genes associated with response to certain therapies (e.g., anti-TNF therapy) may cluster (e.g., form a cluster of genes) in discrete modules on the HI map. The existence of such clusters is associated with the existence of fundamental underlying disease biology. In some embodiments, a classifier is derived from signature genes selected from the cluster of genes on the HI map. Accordingly, in some embodiments, a classifier is derived from a cluster of genes associated with response to anti-TNF therapy on a human interactome map.

In some embodiments, genes associated with response to certain therapies exhibit certain topological properties when mapped onto a human interactome map. For example, in some embodiments, a plurality of genes associated with response to anti-TNF therapy and characterized by their position (e.g., topological properties, e.g., their proximity to one another) on a human interactome map.

In some embodiments, genes associated with response to certain therapies (e.g., anti-TNF therapy) may exist within close proximity to one another on the HI map. Said proximal genes, do not necessarily share fundamental underlying disease biology. That is, in some embodiments, proximal genes do not share significant protein interaction. Accordingly, in some embodiments, the classifier is derived from genes that are proximal on a human interactome map. In some embodiments, the classifier is derived from certain other topological features on a human interactome map.

In some embodiments, genes associated with response to certain therapies (e.g., anti-TNF therapy) may be determined by Diffusion State Distance (DSD) (see Cao, et al., PLOS One, 8(10): e76339 (Oct. 23, 2013), which is incorporated herein by reference for all purposes) when used in combination with the HI map.

In some embodiments, signature genes are selected by (1) ranking genes based on the significance of difference of expression of genes as compared to known responders and known non-responders; (2) selecting genes from the ranked genes and mapping the selected gene s onto a human interactome map; and (3) selecting signature genes from the genes mapped onto the human interactome map. Thus, in some embodiments, signature genes are characterized by the relative ranking of their expression difference in responder vs non-responder subjects or populations.

In some embodiments, signature genes (e.g., selected from the Santolini method, or using various network topological properties including, but not limited to, clustering, proximity and diffusion-based methods) are provided to a probabilistic neural network or other classifier described herein to thereby provide (e.g., “train”) the classifier. In some embodiments, the probabilistic neural network implements the algorithm proposed by D. F. Spechtin “Probabilistic Neural Networks,” Neural Networks, 3(1):109-118 (1990), which is incorporated herein by reference. In some embodiments, the probabilistic neural network is written in the R-statistical language, and knowing a set of observations described by a vector of quantitative variables, classifies observations into a given number of groups (e.g., responders and non-responders). The algorithm is trained with the data set of signature genes taken from known responders and non-responders provides new observations. In some embodiments, the probabilistic neural network is one derived from pnn: Probabilistic neural networks v1.0.1 at The Comprehensive R Archive Network. In some embodiments, signature genes are analyzed according to a Random Forest Model to provide a classifier.

2. Single Nucleotide Polymorphisms

The present disclosure further encompasses an insight that single nucleotide polymorphisms (SNPs) can be identified via RNA sequence data. That is, by comparison of RNA sequence data to a reference human genome, e.g., by mapping RNA sequence data to the GRCh38 human genome. Without wishing to be bound by theory, it is believed that the presence of SNPs that correlate to RNA sequences used in a classifier can facilitate identifying a subpopulation of subjects who respond or do not respond to certain therapies (e.g., anti-TNF therapies). That is, protein products of discriminatory genes or SNP-containing RNAs can be analyzed using network medicine and pathway enrichment analyses. Proteins encoded by discriminatory genes or SNP-containing RNAs included in the classifier can be overlaid on, for example, a map of the human interactome to help identify certain subpopulations of subjects by identifying certain sets of discriminatory genes.

In some embodiments, provided classifiers and methods of using such classifiers, incorporate an assessment related to single nucleotide polymorphisms (SNPs). In some embodiments, the present disclosure provides methods of developing a classifier for stratifying subjects with respect to one or more therapeutic attributes comprising: analyzing sequence data of RNA expressed in subjects representing at least two different categories with respect to at least one of the therapeutic attributes; assessing the presence of one or more single nucleotide polymorphisms (SNPs) from the sequence data; determining the presence of the one or more SNPs correlates with the at least one therapeutic attribute; and including the one or more SNPs in the classifier.

In some embodiments, the present disclosure provides, in a method of developing a classifier for stratifying subjects with respect to one or more therapeutic attributes by analyzing sequence data of RNA expressed in subjects representing at least two different categories with respect to at least one of the therapeutic attributes, the improvement that comprises: assessing presence of one or more single nucleotide polymorphisms (SNPs) from the sequence data; and determining the presence of the one or more SNPs correlates with the at least one therapeutic attribute; and including presence of the one or more SNPs in the classifier.

In some embodiments, one or more SNPs are selected from Table 3.

TABLE 3

chr1.161644258
chr1.2523811
chr11.107967350
chr17.38031857
chr7.1285800042
rs10774624
rs10985070
rs11889341
rs1571878
rs1633360
rs17668708
rs1877030
rs1893592
rs1980422
rs2228145
rs2233424
rs2236668
rs2301888
rs2476601
rs3087243
rs3218251
rs331463
rs34536443
rs34695944
rs4239702
rs4272
rs45475795
rs508970
rs5987194
rs657075
rs6715284
rs706778
rs72634030
rs73013527
rs73194058
rs773125
rs7752903
rs8083786
rs9653442

In some embodiments, SNPs are selected from two or more SNPs from Table 3. In some embodiments, SNPs are selected two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, twenty-eight or more, twenty-nine or more, thirty or more, thirty-one or more, thirty-two or more, thirty-three or more, thirty-four or more, thirty-five or more, thirty-six or more, thirty-seven or more, thirty-eight or more or all 39 SNPs from Table 3.

3. Clinical Characteristics

In some embodiments, a classifier can also incorporate additional information, for example in order to further improve predictive ability of the classifier to identify between responders and non-responders. For example, in some embodiments, a classifier is developed or assessed (e.g., detected) by assessing one or more genes whose expression levels significantly correlate (e.g., in a linear or non-linear manner) to clinical responsiveness or non-responsiveness; and at least one of presence of one or more single nucleotide polymorphisms (SNPs) in an expressed sequence of the one or more genes; or at least one clinical characteristic of the responsive and nonresponsive prior subjects. That is, in some embodiments, a classifier is developed or assessed (e.g., detected) by assessing one or more genes whose expression levels significantly correlate (e.g., in a linear or non-linear manner) to clinical responsiveness or non-responsiveness and the presence of one or more single nucleotide polymorphisms (SNPs) in an expressed sequence of the one or more genes. In some embodiments, a classifier is developed or assessed (e.g., detected) by assessing one or more genes whose expression levels significantly correlate (e.g., in a linear or non-linear manner) to clinical responsiveness or non-responsiveness and at least one clinical characteristic of the responsive and non-responsive prior subjects.

The present disclosure further encompasses an insight that certain clinical characteristics (e.g., B I, gender, age, and the like), can be incorporated into classifiers provided herein. In some embodiments, provided classifiers and methods of using such classifiers, incorporate an assessment related to clinical characteristics. In some embodiments, the present disclosure provides methods of developing a classifier for stratifying subjects with respect to one or more therapeutic attributes comprising: analyzing sequence data of RNA expressed in subjects representing at least two different categories with respect to at least one of the therapeutic attributes; assessing the presence of one or more clinical characteristics; determining that expression related to said clinical characteristics correlate with the at least one therapeutic attribute; and including the one or more clinical characteristics in the classifier.

In some embodiments, at least one clinical characteristic is selected from: body-mass index (BMI), gender, age, race, previous therapy treatment, disease duration, C-reactive protein (CRP) level, presence of anti-cyclic citrullinated peptide, presence of rheumatoid factor, patient global assessment, treatment response rate (e.g., ACR2, ACR50, ACR70), and combinations thereof.

In some embodiments, a clinical characteristic is selected from Table 4.

TABLE 4

Age
Gender at birth
Duration of disease (in years)
Race (included white, Asian, black, mixed race,
Native American, Pacific Islander, and other)
History of fibromyalgia
History of chronic vascular disease (includes
acute coronary syndrome, coronary artery disease,
congestive heart failure, hypertension, myocardial
infarction, peripheral arterial disease, stroke,
unstable angina, cardiac arrest, revascularization
procedure, and ventricular arrhythmia)
History of serious infection that led to hospitalization
(includes infections of bursa or joint, cellulitis, sinusitis,
diverticulitis, sepsis, pneumonia bronchitis gastro
meningitis, urinary tract infection, upper respiratory
infection, and tuberculosis)
History of cancer (includes breast, lung, skin,
lymphoma but excludes non-melanoma skin)
BMI
Smoking status (includes never, previous or current)
Prednisone dose
DMARD dose
C-reactive protein level at baseline
DAS28-CRP at baseline
Swollen 28-joint count at baseline
Tender 28-joint count at baseline
Patient global assessment at baseline
Physician global assessment at baseline
CDAI at baseline
Modified health assessment questionnaire score at baseline
Patient pain assessment at baseline
EULAR response at baseline using DAS28-CRP
(includes poor, moderate or good)
Anti-CCP status (positive or negative)
Anti-CCP titer at baseline
Rheumatoid factor status (positive or negative)
Rheumatoid factor titer at baseline

In some embodiments, clinical characteristics are selected from two or more clinical characteristics from Table 4. In some embodiments, clinical characteristics are selected from two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more or all twenty-six clinical characteristics from Table 4.

4. Validating Classifiers

Alternatively or additionally, in some embodiments, a classifier can be trained in the probabilistic neural network using a cohort of known responders and non-responders using leave-one-out cross or k-fold cross validation. In some embodiments, such a process leaves one sample out (e.g., leave-one-out) of the analysis and trains the classifier based on the remaining samples. In some embodiments, the updated classifier is then used to predict a probability of response for the sample that's left out. In some embodiments, such a process can be repeated iteratively, for example, until all samples have been left out once. In some embodiments, such a process randomly partitions a cohort of known responders and non-responders into k equal sizes groups. Of the k groups, a single group is retained as validation data for testing the model, and the remaining groups are used as training data. Such a process can be repeated k times, with each of the k groups being used exactly once as the validation data. In some embodiments, the outcome is a probability score for each sample in the training set. Such probability scores can correlate with actual response outcome. A Recursive Operating Curves (ROC) can be used to estimate the performance of the classifier. In some embodiments, an Area Under Curve (AUC) of about 0.6 or higher reflects a suitable validated classifier. In some embodiments, a Negative Predictive Value (NPV) of 0.9 reflects a suitable validated classifier. In some embodiments, a classifier can be tested in a completely independent (e.g., blinded) cohort to, for example, confirm the suitability (e.g., using leave-one-out or k-fold cross validation). Accordingly, in some embodiments, provided methods further comprise validating a classifier, for example, by assigning probability of response to a group of known responders and non-responders; and checking the classifier against a blinded group of responders and non-responders. The output of these processes is a trained classifier useful for establishing whether a subject will or will not respond to a particular therapy (e.g., anti-TNF therapy).

In some embodiments, a classifier is established to distinguish between responsive and non-responsive prior subjects who have received a type of therapy, e.g., anti-TNF therapy. This classifier can predict whether a subject will or will not respond to a given therapy. In some embodiments, the response and non-responsive prior subjects suffered from the same disease, disorder, or condition.

In some embodiments, validation of treatment is assessed by monitoring particular clinical characteristics. For example, in some embodiments, treatment response is validated in subjects by statistical analysis of clinical features. In certain embodiments, development, validation, or use of a relevant classifier may involve or have involved assessments of one or more clinical parameters (e.g., of a patient's presentation or status of disease). The present disclosure appreciates that variation may occur in such clinical assessments that may, for example, represent inputs external to the patient (e.g., differences in application of an assessment or interpretation of a patient characteristic or response). The present disclosure provides a solution to this identified problem in providing for patient self-assessment of one or more relevant parameters.

In some embodiments, validation of a classifier comprises statistical analysis of clinical features to analyze changes in clinical characteristics in a patient who has been so classifier by the classifier and received anti-TNF therapy. Such validation methods recognizes that certain subjective measurements of clinical change cannot be quantified compared to methods described herein and involve self-assessment. The present disclosure encompasses an insight that patient self-assessment is not necessarily consistent, but can provide valuable information on treatment response overtime. Such self-assessment response can be used to confirm whether a patient is a true responder or non-responder. For example, statistical analysis of certain clinical characteristics of a cohort of patients can validate the accuracy of the classifier. In some embodiments, statistical analysis of clinical features analyzes changes of one or more of ACR50, ACR70, CDAI LDA, CDAI remission, DAS28-CRP LDA, and DAS28-CRP remission and combinations thereof. In some embodiments, statistical analysis is performed via a Monte Carlo simulation.

In some embodiments, a classifier is validated using a cohort of subjects having previously been treated with anti-TNF therapy, but is independent from the cohort of subjects used to prepare the classifier. In some embodiments, the classifier is updated using gene expression data, SNP data, or clinical characteristics. In some embodiments, a classifier is considered “validated” when 90% or greater of non-responding subjects are predicted with 60% or greater accuracy within the validating cohort.

In some embodiments, the classifier predicts non-responsiveness of subjects with at least 60% accuracy predicting non-responsiveness across a population of at least 100 subjects. In some embodiments, the classifier predicts non-responsiveness of subjects with at least 60% accuracy across a population of at least 150 subjects. In some embodiments, the classifier predicts non-responsiveness of subjects with at least 60% accuracy across a population of at least 170 subjects. In some embodiments, the classifier predicts non-responsiveness of subjects with at least 60% accuracy across a population of at least 200 or more subjects.

In some embodiments, the classifier predicts non-responsiveness of subjects with at least 80% accuracy across a population of at least 100 subjects. In some embodiments, the classifier predicts non-responsiveness of subjects with at least 80% accuracy across a population of at least 150 subjects. In some embodiments, the classifier predicts non-responsiveness of subjects with at least 80% accuracy across a population of at least 170 subjects. In some embodiments, the classifier predicts non-responsiveness of subjects with at least 80% accuracy across a population of at least 200 or more subjects. In some embodiments, the classifier predicts non-responsiveness of subjects with at least 80% accuracy across a population of at least 300 or more subjects. In some embodiments, the classifier predicts non-responsiveness of subjects with at least 80% accuracy across a population of at least 350 or more subjects.

B. Detecting Gene Signature(s) or SNPs

Detecting gene signatures in a subject using a trained classifier may be performed. In other words, by first defining the gene signatures (from the classifier), a variety of methods can be used to determine whether a subject or group of subjects express the established gene signatures. For example, in some embodiments, a practitioner can obtain a blood or tissue sample from the subject prior to administering of therapy, and extract and analyze mRNA profiles from said blood or tissue sample. The analysis of mRNA profiles can be performed by various approaches, including, but not limited to gene arrays, RNA-sequencing, nanostring sequencing, real-time quantitative reverse transcription PCR (qRT-PCR), bead arrays, or enzyme-linked immunosorbent assay (ELISA) and combinations thereof. Accordingly, in some embodiments, the present disclosure provides methods of determining whether a subject is classified as a responder or non-responder, comprising measuring gene expression by at least one of a microarray, RNA sequencing, real-time quantitative reverse transcription PCR (qRT-PCR), bead array, and ELISA and combinations thereof. In some embodiments, the present disclosure provides methods of determining whether a subject is classified as a responder or non-responder comprising measuring gene expression of a subject by RNA sequencing (e.g., RNAseq).

The present disclosure further encompasses an insight that single nucleotide polymorphisms (SNPs) can be identified via RNA sequence data. That is, by comparison of RNA sequence data to a reference human genome, e.g., by mapping RNA sequence data to the GRCh38 human genome. Without wishing to be bound by theory, it is believed that the presence of SNPs that correlate to RNA sequences used in the classifier can facilitate identifying a subpopulation of subjects who respond or do not respond to certain therapies (e.g., anti-TNF therapies). That is, protein products of the discriminatory genes and SNP-containing RNAs can be analyzed using network medicine and pathway enrichment analyses. The proteins encoded by the discriminatory genes and SNP-containing RNAs included in the classifier can be overlaid on, for example, a map of the human interactome to help identify certain subpopulations of subjects by identifying certain sets of discriminatory genes.

In some embodiments, gene expression is measured by subtracting background data, correcting for batch effects, and dividing by mean expression of housekeeping genes. See Eisenberg & Levanon, “Human housekeeping genes, revisited,” Trends in Genetics, 29(10):569-574 (October 2013), which is incorporated herein by reference for all purposes. In the context of microarray data analysis, background subtraction refers to subtracting the average fluorescent signal arising from probe features on a chip not complimentary to any mRNA sequence, e.g., signals that arise from non-specific binding, from the fluorescence signal intensity of each probe feature. The background subtraction can be performed with different software packages, such as Affymetrix Gene Expression Console. Housekeeping genes are involved in basic cell maintenance and, therefore, are expected to maintain constant expression levels in all cells and conditions. The expression level of genes of interest, e.g., those in the response signature, can be normalized by dividing the expression level by the average expression level across a group of selected housekeeping genes. This housekeeping gene normalization procedure calibrates the gene expression level for experimental variability. Further, normalization methods such as robust multi-array average (“RMA”) correct for variability across different batches of microarrays, are available in R packages recommended by either Illumina or Affymetrix platforms. The normalized data is log transformed, and probes with low detection rates across samples are removed. Furthermore, probes with no available genes symbol or Entrez ID are removed from the analysis.

In some embodiments, the present disclosure provides a kit comprising a classifier established to distinguish between responsive and non-responsive prior subjects who have received anti-TNF therapy.

C. Using Classifiers

1. Patient Stratification

Among other things, the present disclosure provides technologies for predicting responsiveness to anti-TNF therapies. In some embodiments, provided technologies exhibit consistency or accuracy across cohorts superior to other methodologies.

Thus, the present disclosure provides technologies for patient stratification, defining or distinguishing between responder and non-responder populations. For example, in some embodiments, the present disclosure provides methods for treating subjects with anti-TNF therapy, which methods, in some embodiments, comprise: administering the anti-TNF therapy to subjects who have been determined to be responsive via a classifier established to distinguish between responsive and non-responsive prior subjects who have received the anti-TNF therapy.

In some embodiments, the present disclosure provides methods of developing a classifier for stratifying subjects with respect to one or more therapeutic attributes comprising: analyzing sequence data of RNA expressed in subjects representing at least two different categories with respect to at least one of the therapeutic attributes; assessing the presence of one or more single nucleotide polymorphisms (SNPs) from the sequence data; determining the presence of the one or more SNPs correlates with the at least one therapeutic attribute; and including the one or more SNPs in the classifier.

Classifiers described herein can be used by analyzing gene expression of subjects. In some embodiments, genes of the subject are measured by at least one of a microarray, RNA sequencing real-time quantitative reverse transcription PCR (qRT-PCR), bead array, ELISA, and protein expression and combinations thereof.

2. Therapy Monitoring

Further, the present disclosure provides technologies for monitoring therapy for a given subject or cohort of subjects. As a subject's gene expression level can change overtime, it may be desirable to evaluate a subject at one or more points in time, for example, at specified and or periodic intervals.

In some embodiments, validation of treatment is assessed by monitoring particular clinical characteristics. For example, in some embodiments, treatment response is validated in subjects by statistical analysis of clinical features. In certain embodiments, development, validation, or use of a relevant classifier may involve or have involved assessments of one or more clinical parameters (e.g., of a patient's presentation or status of disease). The present disclosure appreciates that variation may occur in such clinical assessments that may, for example, represent inputs external to the patient (e.g., differences in application of an assessment or interpretation of a patient characteristic or response). The present disclosure provides a solution to this identified problem in providing for patient self-assessment of one or more relevant parameters.

In some embodiments, validation of a classifier comprises statistical analysis of clinical features to analyze changes in clinical characteristics in a patient who has been so classifier by the classifier and received anti-TNF therapy. Such validation methods recognizes that certain subjective measurements of clinical change cannot be quantified compared to methods described herein and involve self-assessment. The present disclosure encompasses an insight that patient self-assessment is not necessarily consistent but can provide valuable information on treatment response overtime. Such self-assessment response can be used to confirm whether a patient is a true responder or non-responder. For example, statistical analysis of certain clinical characteristics of a cohort of patients can validate the accuracy of the classifier. In some embodiments, statistical analysis of clinical features analyzes changes of one or more of ACR50, ACR70, CDAI LDA, CDAI remission, DAS28-CRP LDA, and DAS28-CRP remission and combinations thereof. In some embodiments, statistical analysis is performed via a Monte Carlo simulation.

In some embodiments, repeated monitoring under time permits or achieves detection of one or more changes in a subject's gene expression profile or characteristics that may impact ongoing treatment regimens. In some embodiments, a change is detected in response to which particular therapy administered to the subject is continued, is altered, or is suspended. In some embodiments, therapy may be altered, for example, by increasing or decreasing frequency or amount of administration of one or more agents or treatments with which the subject is already being treated. Alternatively or additionally, in some embodiments, therapy may be altered by addition of therapy with one or more new agents or treatments. In some embodiments, therapy may be altered by suspension or cessation of one or more particular agents or treatments.

To give but one example, if a subject is initially classified as responsive (because the subject's gene expression was determined, via classifier, to be associated with a disease, disorder, or condition), a given anti-TNF therapy can then be administered. At a given interval (e.g., every six months, every year, etc.), the subject can be tested again to ensure that they still qualify as “responsive” to a given anti-TNF therapy. In the event the gene expression levels for a given subject change over time, and the subject no longer expresses genes associated with the disease, disorder, or condition, or now expresses genes associated with non-responsiveness, the subject's therapy can be altered to suit the change in gene expression.

Accordingly, in some embodiments, the present disclosure provides methods of administering therapy to a subject previously established via classifier as responsive with anti-TNF therapy.

In some embodiments, the present disclosure provides methods further comprising determining, prior to the administering, that a subject is not a responder via a classifier; and administering a therapy alternative to anti-TNF therapy.

In some embodiments, genes of the subject are measured by at least one of a microarray, RNA sequencing, real-time quantitative reverse transcription PCR (qRT-PCR), bead array, ELISA, and protein expression and combinations thereof.

In some embodiments, the subject suffers from a disease, disorder, or condition selected from rheumatoid arthritis, psoriatic arthritis, ankylosing spondylitis, Crohn's disease, ulcerative colitis, chronic psoriasis, hidradenitis suppurativa, multiple sclerosis, and juvenile idiopathic arthritis and combinations thereof.

In some embodiments, the anti-TNF therapy is or comprises administration of infliximab, adalimumab, etanercept, cirtolizumab pegol, golilumab, or biosimilars thereof and combinations thereof. In some embodiments, the anti-TNF therapy is or comprises administration of infliximab or adalimumab.

In some embodiments, the responsive and non-responsive prior subjects suffered from the same disease, disorder, or condition.

In some embodiments, the subjects to whom the anti-TNF therapy is administered are suffering from the same disease, disorder or condition as the prior responsive and non-responsive prior subjects.

In some embodiments, the disease, disorder, or condition is selected from rheumatoid arthritis, psoriatic arthritis, ankylosing spondylitis, Crohn's disease, ulcerative colitis, chronic psoriasis, hidradenitis suppurativa, multiple sclerosis, and juvenile idiopathic arthritis and combinations thereof.

In some embodiments, the disease, disorder, or condition is rheumatoid arthritis.

In some embodiments, the disease, disorder, or condition is ulcerative colitis.

D. Methods of Treatment

In some embodiments, a subject or population with respect to which anti-TNF therapy is administered, or from which anti-TNF therapy is withheld (or alternative therapy is administered) is one that is determined to exhibit a particular expression level one or more genes, and in some cases for a plurality of genes. In some embodiments, one or more genes is determined to have an expression level below a particular threshold; alternatively or additionally, in some embodiments, one or more genes is determined to have an expression level below a particular threshold. In some embodiments, a particular set of genes is determined to have a pattern of expression in which each is assessed relative to a particular threshold (and, e.g., is determined to be above, below, or comparable with such threshold).

In some embodiments, the present disclosure provides methods of treating subjects suffering from a disease, disorder, or condition comprising administering an alternative to anti-TNF therapy to a subject that has been determined to exhibit less than a particular expression level of one or more genes.

In some embodiments, the present disclosure provides methods of administering the anti-TNF therapy to subjects who have been determined to be responsive via a classifier established to distinguish between responsive and non-responsive prior subjects who have received the anti-TNF therapy (e.g., wherein the classifier has been established, through retrospective analysis, to distinguish between those who did vs those who did not respond to anti-TNF therapy that they received); wherein the classifier that is developed by assessing: one or more genes whose expression levels significantly correlate (e.g., in a linear or non-linear manner) to clinical responsiveness or non-responsiveness; and at least one of: presence of one or more single nucleotide polymorphisms (SNPs) in an expressed sequence; and at least one clinical characteristic of the responsive and non-responsive prior subjects.

TNF-mediated disorders are currently treated by inhibition of TNF, and in particular by administration of an anti-TNF agent (e.g., by anti-TNF therapy). Examples of anti-TNF agents approved for use in the United States include monoclonal antibodies such as adalimumab (Humira®), certolizumab pegol (Cimzia®), infliximab (Remicade®), and decoy circulating receptor fusion proteins such as etanercept (Enbrel®). These agents are currently approved for use in treatment of indications, according to dosing regimens, as set forth in Table 5.

TABLE 5

| | | Certolizumab | | | | |
|---|---|---|---|---|---|---|
| Indication | Adalimumab¹ | Pegol¹ | Infliximab² | Etanercept¹ | Golimumab¹ | Golimumab² |
| Juvenile | 10 kg (22 | N/A | N/A | 0.8 mg/kg | N/A | N/A |
| Idiopathic | lbs) to <15 kg | | | weekly, with a | | |
| Arthritis | (33 lbs): | | | maximum of | | |
| | 10 mg | | | 50 mg per | | |
| | every | | | week | | |
| | other | | | | | |
| | week | | | | | |
| | 15 kg (33 | | | | | |
| | lbs) to <30 | | | | | |
| | kg (66 | | | | | |
| | lbs): 20 | | | | | |
| | mg every | | | | | |
| | other | | | | | |
| | week | | | | | |
| | ≥30 kg | | | | | |
| | (66 lbs): | | | | | |
| | 40 mg | | | | | |
| | every | | | | | |
| | other | | | | | |
| | week | | | | | |
| Psoriatic | 40 mg every | 400 mg | 5 mg/kg at 0, | 50 mg once | 50 mg | N/A |
| Arthritis | other week | initially and at | 2 and 6 | weekly with | administered | |
| | | week 2 and 4, | weeks, then | or without | by | |
| | | followed by | every 8 weeks | methotrexate | subcutaneous | |
| | | 200 mg every | | | injection once | |
| | | other week; | | | a month | |
| | | for | | | | |
| | | maintenance | | | | |
| | | dosing, 400 | | | | |
| | | mg every 4 | | | | |
| | | weeks | | | | |
| Rheumatoid | 40 mg every | 400 mg | In conjunction | 50 mg once | 50 mg once a | 2 mg/kg |
| Arthritis | other week | initially and at | with | weekly with | month | intravenous |
| | | Weeks 2 and | methotrexate, | or without | | infusion over |
| | | 4, followed by | 3 mg/kg at 0, | methotrexate | | 30 minutes at |
| | | 200 mg every | 2 and 6 | | | weeks 0 and |
| | | other week; | weeks, then | | | 4, then every |
| | | for | every 8 weeks | | | 8 weeks |
| | | maintenance | | | | |
| | | dosing, 400 | | | | |
| | | mg every 4 | | | | |
| | | weeks | | | | |
| Ankylosing | 40 mg every | 400 mg (given | 5 mg/kg at 0, | 50 mg once | 50 mg | N/A |
| Spondylitis | other week | as 2 | 2 and 6 | weekly | administered | |
| | | subcutaneous | weeks, then | | by | |
| | | injections of | every 6 weeks | | subcutaneous | |
| | | 200 mg each) | | | injection once | |
| | | initially and at | | | a month | |
| | | weeks 2 and | | | | |
| | | 4, followed by | | | | |
| | | 200 mg every | | | | |
| | | other week or | | | | |
| | | 400 mg every | | | | |
| | | 4 weeks | | | | |
| Adult | Initial | 400 mg | 5 mg/kg at 0, | N/A | N/A | N/A |
| Crohn's | dose | initially | 2 and 6 | | | |
| Disease | (Day 1): | and at | weeks, then | | | |
| | 160 mg | Weeks 2 | every 8 | | | |
| | Second | and 4 | weeks. | | | |
| | dose two | Continue | | | | |
| | weeks | with 400 | | | | |
| | later (Day | mg every | | | | |
| | 15): 80 | four | | | | |
| | mg | weeks | | | | |
| | Two | | | | | |
| | weeks | | | | | |
| | later (Day | | | | | |
| | 29): | | | | | |
| | Begin a | | | | | |
| | maintenance | | | | | |
| | dose | | | | | |
| | of 40 mg | | | | | |
| | every | | | | | |
| | other | | | | | |
| | week | | | | | |
| Pediatric | 17 kg (37 lbs) | N/A | 5 mg/kg at 0, | N/A | N/A | N/A |
| Crohn's | to <40 kg (88 | | 2 and 6 | | | |
| Disease | lbs): | | weeks, then | | | |
| | Initial | | every 8 | | | |
| | dose | | weeks. | | | |
| | (Day 1): | | | | | |
| | 80 mg | | | | | |
| | Second | | | | | |
| | dose two | | | | | |
| | weeks | | | | | |
| | later (Day | | | | | |
| | 15): 40 | | | | | |
| | mg | | | | | |
| | Two | | | | | |
| | weeks | | | | | |
| | later (Day | | | | | |
| | 29): | | | | | |
| | Begin a | | | | | |
| | maintenance | | | | | |
| | dose | | | | | |
| | of 20 mg | | | | | |
| | every | | | | | |
| | other | | | | | |
| | week | | | | | |
| | ≥40 kg (88 | | | | | |
| | lbs): | | | | | |
| | Initial | | | | | |
| | dose | | | | | |
| | (Day 1): | | | | | |
| | 160 mg | | | | | |
| | Second | | | | | |
| | dose two | | | | | |
| | weeks | | | | | |
| | later (Day | | | | | |
| | 15): 80 | | | | | |
| | mg | | | | | |
| | Two | | | | | |
| | weeks | | | | | |
| | later (Day | | | | | |
| | 29): | | | | | |
| | Begin a | | | | | |
| | maintenance | | | | | |
| | dose | | | | | |
| | of 40 mg | | | | | |
| | every | | | | | |
| | other | | | | | |
| | week | | | | | |
| Ulcerative | Initial | N/A | 5 mg/kg at 0, | N/A | N/A | N/A |
| Colitis | dose | | 2 and 6 | | | |
| | (Day 1): | | weeks, then | | | |
| | 160 mg | | every 8 | | | |
| | Second | | weeks. | | | |
| | dose two | | | | | |
| | weeks | | | | | |
| | later (Day | | | | | |
| | 15): 80 | | | | | |
| | mg | | | | | |
| | Two | | | | | |
| | weeks | | | | | |
| | later (Day | | | | | |
| | 29): | | | | | |
| | Begin a | | | | | |
| | maintenance | | | | | |
| | dose | | | | | |
| | of 40 mg | | | | | |
| | every | | | | | |
| | other | | | | | |
| | week | | | | | |
| Plaque | 80 mg initial | N/A | N/A | 50 mg twice | N/A | N/A |
| Psoriasis | dose; 40 mg | | | weekly for 3 | | |
| | every other | | | months, | | |
| | week | | | followed by | | |
| | beginning one | | | 50 mg once | | |
| | week after | | | weekly | | |
| | initial dose | | | | | |
| Hidradenitis | Initial | N/A | N/A | N/A | N/A | N/A |
| Suppurativa | dose | | | | | |
| | (Day 1): | | | | | |
| | 160 mg | | | | | |
| | Second | | | | | |
| | dose two | | | | | |
| | weeks | | | | | |
| | later (Day | | | | | |
| | 15): 80 | | | | | |
| | mg | | | | | |
| | Third | | | | | |
| | dose | | | | | |
| | (Day 29) | | | | | |
| | and | | | | | |
| | subsequent | | | | | |
| | doses: | | | | | |
| | 40 mg | | | | | |
| | every | | | | | |
| | week | | | | | |
| Uveitis | 80 mg initial | N/A | N/A | N/A | N/A | N/A |
| | dose; 40 mg | | | | | |
| | every other | | | | | |
| | week | | | | | |
| | beginning one | | | | | |
| | week after | | | | | |
| | initial dose | | | | | |

¹Administered by subcutaneous injection.
²Administered by intravenous infusion.

The present disclosure provides technologies relevant to anti-TNF therapy, including those therapeutic regimens as set forth in Table 5. In some embodiments, the anti-TNF therapy is or comprises administration of infliximab (Remicade®), adalimumab (Humira®), certolizumab pegol (Cimzia®), etanercept (Enbrel®), or biosimilars thereof. In some embodiments, the anti-TNF therapy is or comprises administration of infliximab (Remicade®) or adalimumab (Humira®) and combinations thereof. In some embodiments, the anti-TNF therapy is or comprises administration of infliximab (Remicade®). In some embodiments, the anti-TNF therapy is or comprises administration of adalimumab (Humira®).

In some embodiments, the anti-TNF therapy is or comprises administration of a biosimilar anti-TNF agent. In some embodiments, the anti-TNF agent is selected from infliximab biosimilars such as CT-P13, BOW015, SB2, Inflectra®, Renflexis®, and Ixifi™, adalimumab biosimilars such as ABP 501 (AMGEVITA™), Adfrar, and Hulio™ and etanercept biosimilars such as HD203, SB4 (Benepali®), GP2015, Erelzi®, and Intacept and combinations thereof.

In some embodiments, treatment of, for example, juvenile idiopathic arthritis, psoriatic arthritis, rheumatoid arthritis, ankylosing spondylitis, pediatric Crohn's Disease, ulcerative colitis, plaque psoriasis, hidradenitis suppurativa, and uveitis comprises a dosing regimen of an anti-TNF agent in Table 5. In some embodiments, the anti-TNF agent comprises, for example, adalimumab in Table 5. In some embodiments, dosing regimen for adalimumab comprises, for example, an initial dose of up to 160 mg or more. In some embodiments, dosing regimen for adalimumab comprises, for example, a second dose of up to 80 mg or more. In some embodiments, dosing regimen for adalimumab comprises, for example, a maintenance dose of up to 40 mg or more every other week. In some embodiments, the anti-TNF agent comprises, for example, certolizumab pegol in Table 5. In some embodiments, dosing regimen for certolizumab pegol comprises, for example, a first initial dose up to 400 mg or more. In some embodiments, dosing regimen for certolizumab pegol comprises, for example, a second initial dose up to 400 mg or more at week 2. In some embodiments, dosing regimen for certolizumab pegol comprises, for example, a third initial dose up to 400 mg or more at week 4. In some embodiments, dosing regimen for certolizumab pegol comprises, for example, a maintenance dose of up to 200 mg or more every other week or a maintenance dose of up to 400 mg or more every four weeks. In some embodiments, the anti-TNF agent comprises, for example, infliximab in Table 5. In some embodiments, dosing regimen for infliximab comprises, for example, a first initial dose of up to 5 mg/kg or more. In some embodiments, dosing regimen for infliximab comprises, for example, a second initial dose of up to 5 mg/kg or more at week 2. In some embodiments, dosing regimen for infliximab comprises, for example, a third initial dose of up to 5 mg/kg or more at week 6. In some embodiments, dosing regimen for infliximab comprises, for example, a maintenance dose of up to 5 mg/kg or more every 6 weeks or every 8 weeks. In some embodiments, the anti-TNF agent comprises, for example, etanercept in Table 5. In some embodiments, dosing regimen for etanercept comprises, for example, initial doses of up to 50 mg or more twice weekly for three months. In some embodiments, dosing regimen for etanercept comprises, for example, a maintenance dose of up to 50 mg or more every week. In some embodiments, the anti-TNF agent comprises, for example, golimumab in Table 5. In some embodiments, dosing regimen for golimumab comprises, for example, a dose of up to 50 mg or more every month. In some embodiments, dosing regimen for golimumab comprises, for example, a first initial dose of up to 2 mg/kg. In some embodiments, dosing regimen for golimumab comprises, for example, a second initial dose of up to 2 mg/kg or more at week 2. In some embodiments, dosing regimen for golimumab comprises, for example, a maintenance dose of up to 2 mg/kg or more every 8 weeks.

In some embodiments, the present disclosure provides methods of treating subjects suffering from an autoimmune disorder, the method comprising: administering an anti-TNF therapy to subjects who have been determined to be responsive via a classifier established to distinguish between responsive and non-responsive prior subjects in a cohort who have received the anti-TNF therapy; wherein the classifier is developed by assessing: one or more genes whose expression levels significantly correlate (e.g., in a linear or non-linear manner) to clinical responsiveness or non-responsiveness; at least one of: presence of one or more single nucleotide polymorphisms (SNPs) in an expressed sequence of the one or more genes; or at least one clinical characteristic of the responsive and non-responsive prior subjects; and wherein the classifier is validated by an independent cohort than the cohort who have received the anti-TNF therapy.

In some embodiments, the subject has been previously administered the anti-TNF therapy. In some embodiments, the subject has been administered the anti-TNF therapy at least one, at least two, at least three, at least four, at least five, or at least six months prior to said administering.

In some embodiments, data derived from subjects in the cohort who have received the anti-TNF therapy is of one type (e.g., microarray, RNAseq, etc.), and the data used to validate the classifier in the independent cohort is derived from a different type (e.g., microarray, RNAseq). Accordingly, some embodiments, the classifier is established using microarray analysis derived from the responsive and non-responsive prior subjects. In some embodiments, the classifier is validated using RNAseq data derived from the independent cohort.

E. Diseases, Disorders or Conditions

In general, provided disclosures are useful in any context in which administration of anti-TNF therapy is contemplated or implemented. In some embodiments, provided technologies are useful in the diagnosis or treatment of subjects suffering from a disease, disorder, or condition associated with aberrant (e.g., elevated) TNF expression or activity. In some embodiments, provided technologies are useful in monitoring subjects who are receiving or have received anti-TNF therapy. In some embodiments, provided technologies identify whether a subject will or will not respond to a given anti-TNF therapy. In some embodiments, the provided technologies identify whether a subject will develop resistance to a given anti-TNF therapy.

Accordingly, the present disclosure provides technologies relevant to treatment of the various disorders related to TNF, including those listed in Table 5. In some embodiments, a subject is suffering from a disease, disorder, or condition selected from rheumatoid arthritis, psoriatic arthritis, ankylosing spondylitis, Crohn's disease (adult or pediatric), ulcerative colitis, inflammatory bowel disease, chronic psoriasis, plaque psoriasis, hidradenitis suppurativa, asthma, uveitis, and juvenile idiopathic arthritis and combinations thereof. In some embodiments, the disease, disorder, or condition is rheumatoid arthritis. In some embodiments, the disease, disorder, or condition is psoriatic arthritis. In some embodiments, the disease, disorder, or condition is ankylosing spondylitis. In some embodiments, the disease, disorder, or condition is Crohn's disease. In some embodiments, the disease, disorder, or condition is adult Crohn's disease. In some embodiments, the disease, disorder, or condition is pediatric Crohn's disease. In some embodiments, the disease, disorder, or condition is inflammatory bowel disease. In some embodiments, the disease, disorder, or condition is ulcerative colitis. In some embodiments, the disease, disorder, or condition is chronic psoriasis. In some embodiments, the disease, disorder, or condition is plaque psoriasis. In some embodiments, the disease, disorder, or condition is hidradenitis suppurativa. In some embodiments, the disease, disorder, or condition is asthma. In some embodiments, the disease, disorder, or condition is uveitis. In some embodiments, the disease, disorder, or condition is juvenile idiopathic arthritis.

In some embodiments, the disease, disorder or condition is granuloma annulare, necrobiosis lipoidica, hiradenitis suppurativa, pyoderma gangrenossum, Sweet's syndrome, subcorneal pustular dermatosis, systemic lupus erythematosus, scleroderma, dermatomyositis, Behcet's disease, acute/chronic graft versus host disease, pityriasis rubra pilaris, Sjorgren's syndrome, Wegener's granulomatosis, polymyalgia rheumatic, dermatomyositis, and pyoderma gangrenosum and combinations thereof.

Further, as noted, the present disclosure provides technologies that allow practitioners to reliably and consistently predict response in a cohort of subjects. In particular, for example, the rate of response for some anti-TNF therapies is less than 35% within a given cohort of subjects. The provided technologies allow for prediction of greater than 65% accuracy within a cohort of subjects a response rate (e.g., whether certain subjects will or will not respond to a given therapy). In some embodiments, the methods and systems described herein predict 65% or greater the subjects that are non-responders (e.g., will not respond to anti-TNF therapy) within a given cohort. In some embodiments, the methods and systems described herein predict 70% or greater the subjects that are non-responders (e.g., will not respond to anti-TNF therapy) within a given cohort. In some embodiments, the methods and systems described herein predict 80% or greater the subjects that are non-responders (e.g., will not respond to anti-TNF therapy) within a given cohort. In some embodiments, the methods and systems described herein predict 90% or greater the subjects that are non-responders (e.g., will not respond to anti-TNF therapy) within a given cohort. In some embodiments, the methods and systems described herein predict 100% the subjects that are non-responders (e.g., will not respond to anti-TNF therapy) within a given cohort.

### Computer Control Systems

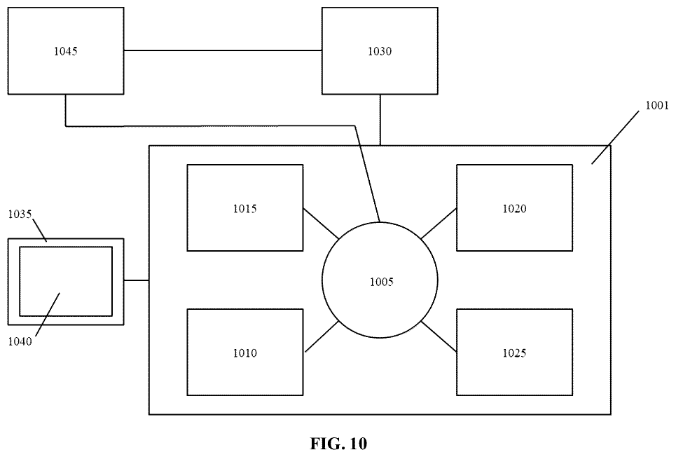

The present disclosure provides computer control systems that are programmed to implement methods of the disclosure. FIG. 10 shows a computer system 1001 that is programmed or otherwise configured to generate or develop autoantibody profile or compare autoantibodies with the profile of the specific immune response. The computer system 1001 can regulate various aspects of the present disclosure, such as, for example, receive or generate sequence reads, correlate sequences to specific epitopes or autoantibodies, output a result for the user as to the presence of an autoantibody or profile, or an expected progression of a disease. The computer system 1001 can be an electronic device of a user or a computer system that is remotely located with respect to the electronic device. The electronic device can be a mobile electronic device.

The computer system 1001 includes a central processing unit (CPU, also “processor” and “computer processor” herein) 1005, which can be a single core or multi core processor, or a plurality of processors for parallel processing. The computer system 1001 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage or electronic display adapters. The memory 1010, storage unit 1015, interface 1020 and peripheral devices 1025 are in communication with the CPU 1005 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020. The network 1030 can be the Internet, an internet or extranet, or an intranet or extranet that is in communication with the Internet. The network 1030 in some cases is a telecommunication or data network. The network 1030 can include one or more computer servers, which can enable distributed computing, such as cloud computing. The network 1030, in some cases with the aid of the computer system 1001, can implement a peer-to-peer network, which may enable devices coupled to the computer system 1001 to behave as a client or a server.

The CPU 1005 can execute a sequence of machine-readable instructions, which can be embodied in a program or software. The instructions may be stored in a memory location, such as the memory 1010. The instructions can be directed to the CPU 1005, which can subsequently program or otherwise configure the CPU 1005 to implement methods of the present disclosure. Examples of operations performed by the CPU 1005 can include fetch, decode, execute, and writeback.

The CPU 1005 can be part of a circuit, such as an integrated circuit. One or more other components of the system 1001 can be included in the circuit. In some cases, the circuit is an application specific integrated circuit (ASIC).

The storage unit 1015 can store files, such as drivers, libraries and saved programs. The storage unit 1015 can store user data, e.g., user preferences and user programs. The computer system 1001 in some cases can include one or more additional data storage units that are external to the computer system 1001, such as located on a remote server that is in communication with the computer system 1001 through an intranet or the Internet.

The computer system 1001 can communicate with one or more remote computer systems through the network 1030. For instance, the computer system 1001 can communicate with a remote computer system of a user. Examples of remote computer systems include personal computers (e.g., portable PC), slate or tablet PC's (e.g., Apple® iPad, Samsung® Galaxy Tab), telephones, Smartphones (e.g., Apple® iPhone, Android-enabled device, Blackberry®), or personal digital assistants. The user can access the computer system 1001 via the network 1030.

Methods as described herein can be implemented by way of machine (e.g., computer processor) executable code stored on an electronic storage location of the computer system 1001, such as, for example, on the memory 1010 or electronic storage unit 1015. The machine executable or machine-readable code can be provided in the form of software. During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 1001, can be embodied in programming. Various aspects of the technology may be thought of as “products” or “articles of manufacture” typically in the form of machine (or processor) executable code or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code or data. Many of these forms of computer readable media may be involved in carrying one or more sequences of one or more instructions to a processor for execution.

The computer system 1001 can include or be in communication with an electronic display 1035 that comprises a user interface (UI) 1040 for providing, for example, selecting autoantibodies for analysis, interacting with graphs correlating autoantibodies to specific generated profiles. Examples of UI's include, without limitation, a graphical user interface (GUI) and web-based user interface.

Methods and systems of the present disclosure can be implemented byway of one or more algorithms. An algorithm can be implemented by way of software upon execution by the central processing unit 1005. The algorithm can, for example, calculate statistics measurements to identify autoantibodies and generate profiles or predict efficacy and toxicity of a treatment.

### EXAMPLES

### Example 1—a Molecular Signature Response Classifier to Predict Inadequate Response to Tumor Necrosis Factor-Alpha Inhibitors in Rheumatoid Arthritis

Rheumatoid arthritis (RA) is an autoimmune disease characterized by chronic inflammation that causes joint destruction. Following inadequate response to synthetic disease modifying anti-rheumatic drugs (csDMARDs) such as methotrexate, clinical guidelines suggest one of many targeted therapies with comparable efficacies and safety profiles including tumor necrosis factor-a inhibitors (TNFi), IL-6 inhibitors, Janus kinase (JAK) inhibitors, and B or T cell modulators. The abundance of treatment options underscores the need for precision medicine in rheumatology. Because clinical guidelines do not recommend one treatment over another, therapy selection is often driven by administrative directives and TNFi therapies remain the prevailing treatment in nearly 90% of RA patients. Matching each patient with the right targeted therapy to reach treat-to-target goals of low disease activity (LDA) or remission is a critical unmet medical need in RA.

A subset of RA patients have an adequate response to TNFi treatment: 50-70% achieve ACR20, 30-40% achieve ACR50, and 15-25% achieve ACR70 response and 10-25% achieve remission. Many studies have attempted to identify biomarkers and develop models to predict response to TNFi therapy before the initiation of treatment. Failure to validate and reproduce the performance of these predictive biomarkers in new patient populations and clinical trials was a typical outcome. Differing characteristics between patient populations, laboratory methods, procedures in generating molecular data, and other biases inherent to single-cohort retrospective blood studies have hindered precision medicine progress not only in rheumatology but in other medical specialties as well.

A blood-based molecular signature test that integrated next generation RNA sequencing data with clinical features to predict the likelihood of an RA patient having an inadequate response to TNFi therapy was developed with a novel network medicine approach to biomarker discovery. Clinical validation of this molecular signature test in a subset of patients from the CERTAIN study revealed that patients with a molecular signature of non-response were unlikely to reach ACR50 at 6 months.

Mapping of disease-related proteins onto the human interactome, a network map of pairwise protein-protein interactions that occur in human cells, has yielded new insights into human disease biology and response to therapy. With the identification of molecular biomarkers involved in RA biology discovered from human interactome analyses, this study demonstrated the TNFi inadequate response prediction performance of a molecular signature response classifier (MRSC) in two prospective observational clinical studies: the CERTAIN study, and NETWORK-004. Validation of the MSRC was performed in a total of 391 blood samples from targeted therapy-naive patients and 113 blood samples from TNFi therapy-exposed patients.

### Methods

Patients

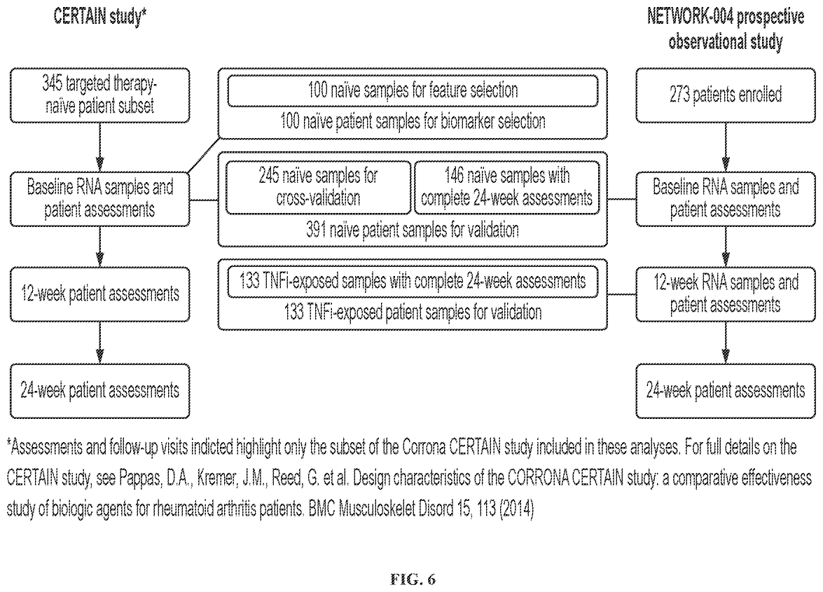

An outline of the study design, Corrona, is described in FIG. 6. The CERTAIN study included: 345 RA patient PAXgene blood samples and clinical measurements, a comparative effectiveness study for RA patients initiating a biologic. The CERTAIN study was nested within the Corrona registry. Institutional Review Board or Ethics Committee approvals were obtained prior to sample collection and study participation, and patients provided informed consent. CERTAIN was a comparative effectiveness study investigating initiators of biologics. For these analyses, samples selected were from patients who were naive to targeted therapies at the time of sample collection and initiated a TNFi therapy. 92% (318/345) of these patients were included in previous classifier training and validation analyses. Consistent with the inclusion criteria of the CERTAIN study, all patients had a Clinical Disease Activity Index (CDAI) greater than ten at the time of biologic therapy initiation. Clinical and molecular data were used for biomarker feature selection (100 patients) and in-cohort cross-validation (245 patients). Patients were randomly allocated to these two separate analyses, irrespective of how each sample was used in previous studies.

The NETWORK-004: Patients were determined by the treating rheumatologist to be candidates for TNFi therapy prior to enrollment. Eligible patients were >18 years of age, had active RA (CDAI >10, swollen joint count >4) and were receiving a stable dose of methotrexate (>15 mg/week) for >10 weeks prior to baseline. Doses of hydroxychloroquine not exceeding 400 mg per day or leflunomide not exceeding 20 mg per day were permitted so long as the dose was stable for at least 4 weeks prior to the baseline visit. Prednisone doses of <10 mg per day were allowable as long as the dose was stable for at least 2 weeks prior to baseline. Use of intra-articular or parenteral corticosteroids <2 weeks prior to the first study procedure was prohibited. The study was approved by the Copernicus Group Independent Review Board (approval #20191082) and local review boards where required. All patients provided written informed consent. Dosage and treatment of TNFi therapies were at the rheumatologist's discretion. At the 3-month follow-up, rheumatologists were permitted to make dosing adjustments if deemed appropriate clinical care. Initiation of a second TNFi therapy resulted in subject withdrawal. The COVID-19 pandemic contributed to a higher level of attrition than initially projected. Of the 273 RA patients enrolled, 168 completed the 24-week study. 146 patients had complete clinical and molecular data and were included in analyses. Information about patients who left the study are available in Supplementary Table Si. PAXgene blood samples collected at the 3-month visits from 113 patients were analyzed as TNFi-exposed samples.

Clinical Evaluation and Response to TNFi Therapies

Feature selection response definition: Clinical outcome metrics such as swollen and tender joint counts, patient and physician disease assessments have inherent variability. To identify a subset of patients in the training cohort who have been assigned the responder and non-responder labels with high confidence, a Monte-Carlo simulation approach was implemented to calculate a confidence outcome score for each patient. The clinical outcomes data for patients with at least 70% concordance between the simulations and the actual reported outcome were considered high confidence. High confidence clinical outcomes for both ACR and EULAR metrics were used for the feature selection.

The CERTAIN study examined baseline RNA sequencing data and clinical assessments to predict response to TNFi therapy at the 3- and 6-month follow-up visits according to ACR, CDAI and DAS28-CRP criteria.

The NETWORK-004 study examined clinical assessments were collected at baseline, 3-month and 6-month visits: 28-joint count for tenderness and swelling, patient global assessment of pain, patient global assessment of disease activity, CDAI score, Health Assessment Questionnaire and C-reactive protein (CRP). Rheumatoid factor (RF) and anti-cyclic citrullinated protein (anti-CCP) antibody serostatus was recorded at baseline. PAXgene RNA blood tubes were collected at all visits. The 3-month follow-up visit RNA sequencing data was used to predict response to TNFi therapy at the 6-month follow-up visit according to the ACR, CDAI and DAS28-CRP criteria.

RNA Preparation and Sequencing Analysis

RNA was extracted from whole blood in PAXgene RNA tubes using MagMax™ for Stabilized Blood PAXgene Tubes RNA Isolation Kit (Thermo Fisher Scientific) per the manufacturer's instructions; 100-1000 ng of RNA was processed using the KAPA RNA HyperPrep Kit with RiboErase (HMIR) Globin. Samples were quantified using Agilent D1000 reagents. Libraries were sequenced to high uniform depth targeting >7 million protein coding reads. The CERTAIN cohort was sequenced using the Illumina NextSeq DX 500 and NovaSeq 6000 instruments. The NETWORK-004 samples were sequenced using the Illumina NovaSeq 6000 instrument using a validated diagnostic assay under Clinical Laboratory Improvement Amendments (CLIA). Sequence data was processed to determine gene expression across the whole genome. To be included in analyses, samples had to have a TapeStation RIN >4, RNA concentration >10 ng/μL, sequencing library yield >10 nM, % perfect basepair index >85, % bases over Phred score 30>75, the mean quality Phred score >30, the median Phred score >25 and a lower quartile Phred score >10 for all bases.

Human Interactome Analysis and Feature Selection

For selection of transcript biomarker features, 100 samples were randomly selected out of the cohort of 345 patients (CERTAIN study). The random forest algorithm was used to rank protein coding transcripts through 96 rounds of 20% cross validation in-silico experiments. Features that were ranked in the top 100 in 70/96 iterations were further analyzed by the human interactome analysis to identify biologically relevant biomarkers. Biomarkers overlapping with the RA disease module on the human interactome³⁵ or possessing a significant number of connections to the disease module were used in the final model. Significance of connections was assessed using the hypergeometric test.

Predictive Classification Model Training and Validation

Samples not included in biomarker feature selection were evaluated. Transcripts identified in this study were integrated with previously described biomarkers using machine learning to re-train a response classification model. To assess model performance, 10-fold cross-validation was conducted using a feedforward artificial neural network. Model building was done using the MLPClassifier package available in Python's machine learning library sklearn.

Statistical Analysis

Statistical analyses were performed using Python 3.7.6 and R version 3.6.1. Continuous data were summarized with mean, standard deviation, median, minimum, maximum, and number of evaluable observations. Categorical variables were summarized with frequency counts and percentages. Confidence intervals (CI) were determined, where appropriate, using the t-distribution for continuous data an exact method for categorical variables. All tests were done in a two-sided setting. Unless otherwise specified, hypothesis testing was performed at the two-sided 0.05 significance level. All attempts were made to limit missing data. No attempt was made to impute missing data.

Results

Identification of a Molecular Signature of Non-Response to TNFi Therapies Using the Human Interactome

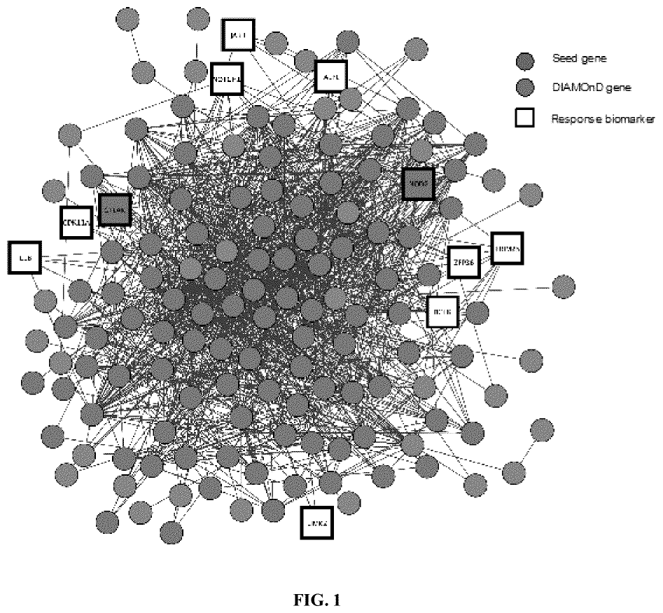

Transcripts predictive of inadequate response to TNFi therapies according to ACR50 and EULAR response definitions (see Methods) at 6 months were determined using machine learning from baseline blood sample data of 100 RA targeted-therapy naive patients randomly selected from the Corrona CERTAIN study. To ensure that transcripts reflected RA disease biology, the proteins encoded by selected transcripts were mapped onto the human interactome map of pairwise protein-protein interactions to identify transcripts that were significantly connected (p-value <0.05) to the RA disease module (FIG. 1). The TNFi therapy response features overlap with to the same network neighborhood of the human interactome consisting of RA disease-associated proteins. These features included proteins with relevance to RA pathobiology, including JAK3 and interleukin-1 beta (IL-1B). The molecular signature of non-response to TNFi therapy included 23 features: 19 RNA transcripts and 4 clinical features (Table 6):

TABLE 6

ALPL
ATRAID
BCL6
CDK11A
CFLAR
COMMD5
GOLGA1
IL1B
IMPDH2
JAK3
KLHDC3
LIMK2
NOD2
NOTCH1
SPINT2
SPON2
STOML2
TRIM25
ZFP36
BMI
Sex
Patient Global
Assessment
Anti-CCP

In-Cohort Validation of the MSRC

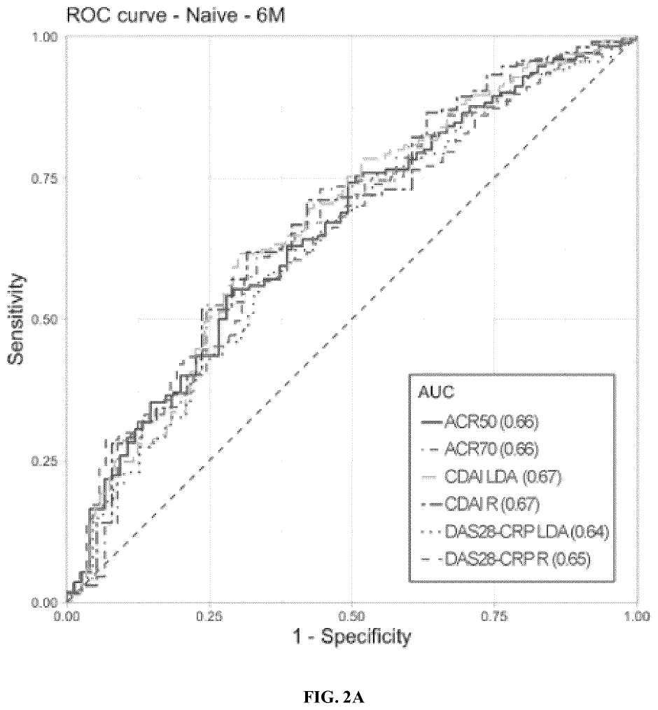

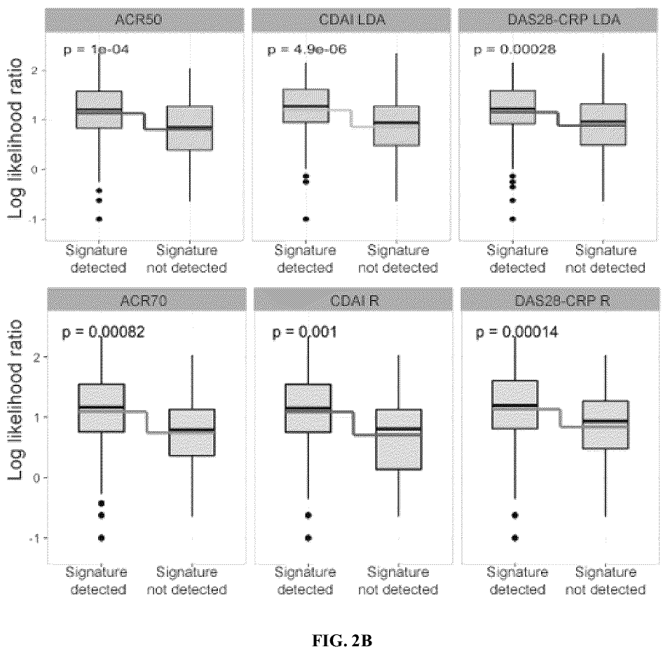

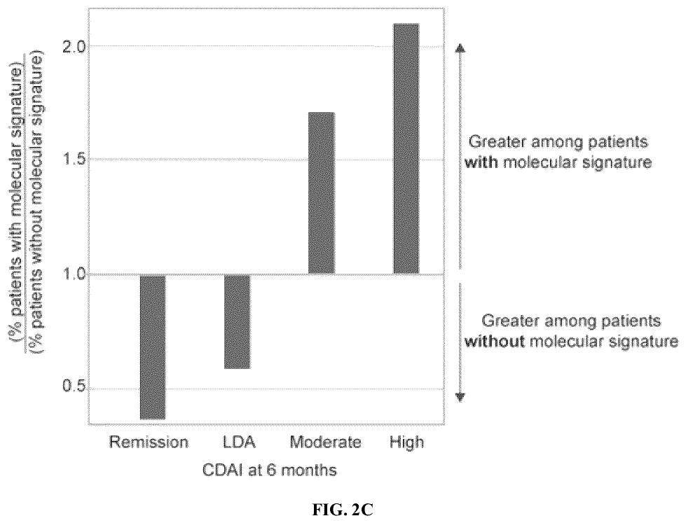

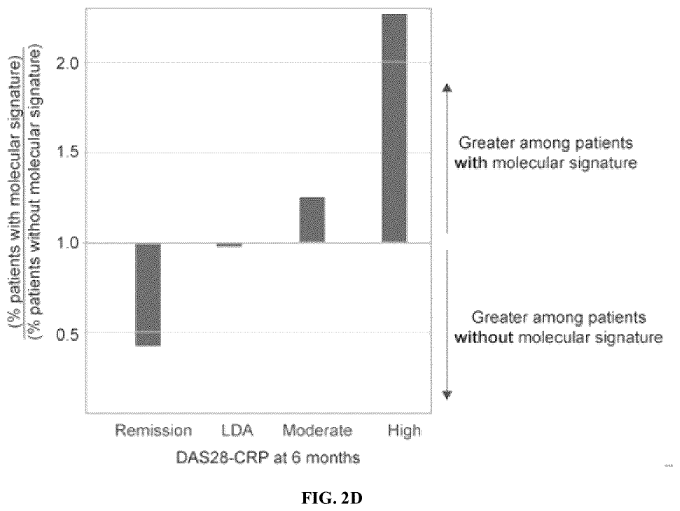

The MSRC was tested through in-cohort cross-validation among baseline blood samples of an independent cohort of 245 patients from the Corrona CERTAIN study who were naive to targeted therapies (Table 7). This resulted in AUC values of 0.63 to 0.67 for ACR50, ACR70, CDAI and DAS28 responses at six months post treatment initiation (FIG. 2A, and Table 8). Significant differences in the log-likelihood ratio of model scores (p<0.001) were observed between patients who did and did not have the molecular signature of non-response (FIG. 211). Furthermore, the proportion of patients who achieved LDA or remission at 6 months per CDAI and DAS28-CRP was greater among those patients who lacked a molecular signature of non-response (FIG. 2C).

TABLE 7

| | Corrona | | Corrona | | NETWORK- | | | |
|---|---|---|---|---|---|---|---|---|
| | CERTAIN | | CERTAIN | | 004 study | | NETWORK- | |
| | study feature | | study cross- | | targeted | | 004 study | |
| | selection | | validation | | therapy-naïve | | TNFi-exposed | |
| Characteristic | (N = 100) | | (N = 245) | | (N = 146) | | (N = 113) | |
| Age (year), mean (SD) | 54 | (12.4) | 55 | (12.3) | 58 | (14.1) | 57 | (14.9) |
| Female, n (%) | 72 | (72.0%) | 179 | (73.1%) | 115 | (78.8%) | 87 | (77.0%) |
| Duration of disease | 1 | (1, 5) | 2 | (1, 6) | 1 | (0, 4.25) | 1 | (0, 4) |
| (year), median (IQR) | | | | | | | | |
| Race, n (%) | | | | | | | | |
| White | 83 | (83.0%) | 213 | (86.9%) | 113 | (77.4%) | 92 | (81.4%) |
| Black | 9 | (9.0%) | 13 | (5.3%) | 16 | (11.0%) | 12 | (10.6%) |
| Other | 8 | (8.0%) | 19 | (7.8%) | 13 | (8.9%) | 9 | (8.0%) |
| CCP positive, n (%) | 62 | (62.0%) | 154 | (62.9%) | 72 | (49.3%) | 54 | (47.8%) |
| RF positive, n (%) | 76 | (76.0%) | 172 | (70.2%) | 55 | (37.7%) | 48 | (54.5%) |
| Prednisone at baseline, | 30 | (30.0%) | 64 | (26.1%) | 33 | (22.6%) | 23 | (20.4%) |
| n (%) | | | | | | | | |
| Prednisone dosage, | 5 | (5, 10) | 5 | (5, 10) | 5 | (5, 9.38) | 5 | (5, 5) |
| median (IQR) | | | | | | | | |
| Current csDMARD, n | | | | | | | | |
| (%) | | | | | | | | |
| Methotrexate | 56 | (56.0%) | 138 | (56.3%) | 113 | (77.4%) | 81 | (71.7%) |
| >2 csDMARDs | 7 | (7.0%) | 42 | (17.1%) | 11 | (7.5%) | 8 | (7.1%) |
| None | 15 | (15.0%) | 37 | (15.1%) | 32 | (22.6%) | 32 | (28.3%) |
| TNFi use, n (%) | | | | | | | | |
| Adalimumab | 36 | (36.0%) | 98 | (40.0%) | 48 | (32.9%) | 40 | (35.4%) |
| Etanercept | 35 | (35.0%) | 76 | (31.0%) | 31 | (21.2%) | 25 | (22.1%) |
| Infliximab | 15 | (15.0%) | 48 | (19.6%) | 18 | (12.3% | 16 | (14.2%) |
| Certolizumab pegol | 10 | (10.0%) | 17 | (6.9%) | 13 | (8.9%) | 7 | (6.2%) |
| Golimumab | 4 | (4.0%) | 6 | (2.4%) | 36 | (24.7%) | 25 | (22.1%) |

TABLE 8

| Cross-validation, naive | AUC | Odds ratio (95% CI; p-value) |
|---|---|---|
| ACR50, 6 months | 0.66 | 3.0 (1.6-5.5; 0.0002) |
| ACR70, 6 months | 0.66 | 3.4 (1.6-7.1; 0.0008) |
| CDAILDA, 6 months | 0.67 | 3.7 (2.2-6.4; <0.0001) |
| CDAI remission, 6 months | 0.67 | 3.4 (1.6-7.6; 0.0014) |
| DAS28-CRP LDA, 6 months | 0.64 | 2.5 (1.5-4.3; 0.0005) |
| DAS28-CRP remission, 6 months | 0.65 | 2.7 (1.6-4.7; 0.0003) |

Validation of the MSRC in a Prospective Observational Clinical Study Among Targeted Treatment Naive Patient Samples

To further validate the ability of the MSRC to predict the likelihood of inadequate response to TNFi therapies, patient samples were prospectively collected in a multi-center observational clinical study. Following clinical and molecular data quality review, 146 patients completed the 24-week study and were included in analyses. These patients were predominantly female (78.8%) and white (80.1%), with a median age of 58 years (Table 7). TNFi therapy choice was at the discretion of the prescribing physician and all five therapeutic options within the class were represented (adalimumab 32.9%, certolizumab pegol 8.9%, etanercept 21.2%, infliximab 12.3% and golimumab 24.7%). A molecular signature of non-response was detected at baseline for 44.5% (65/146) of patients.

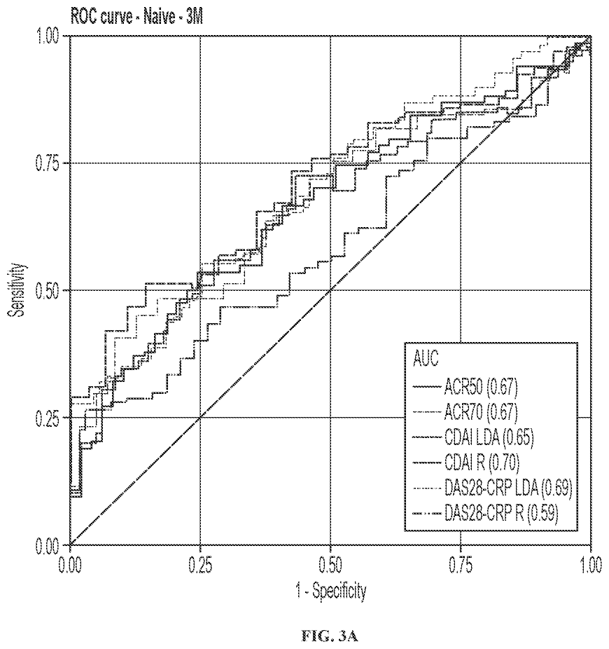

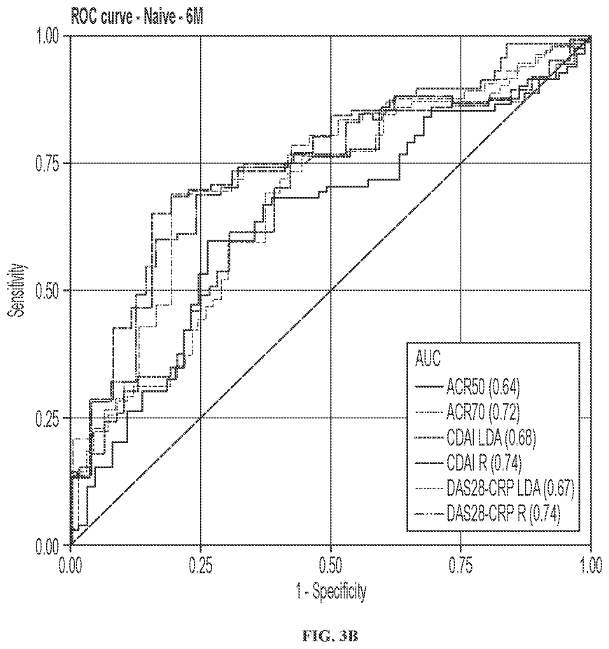

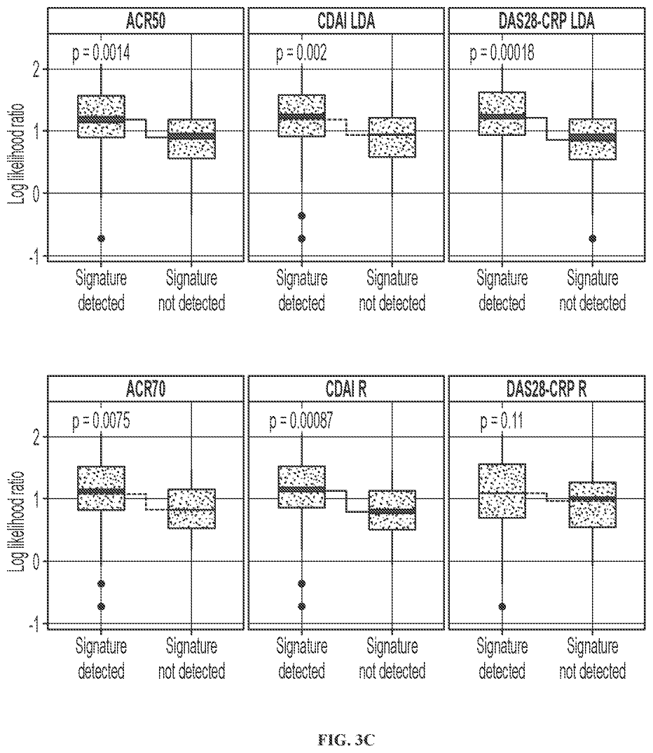

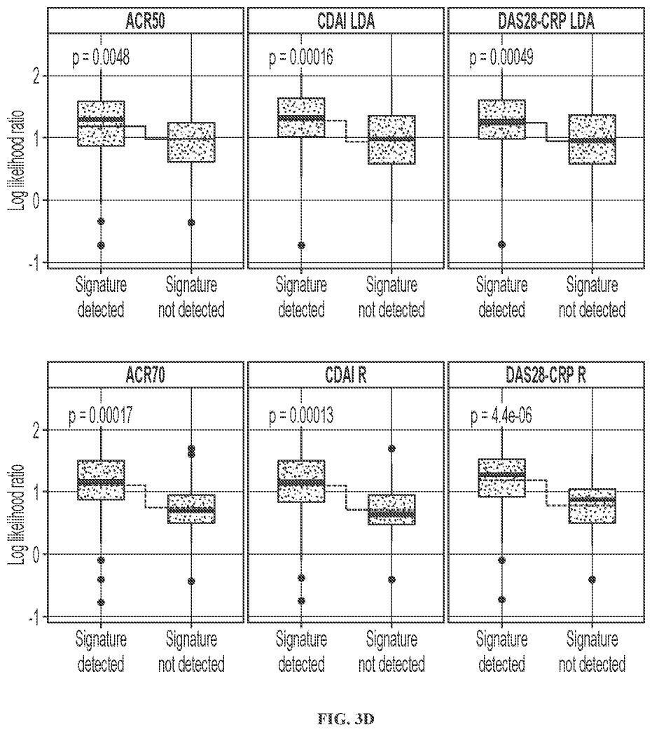

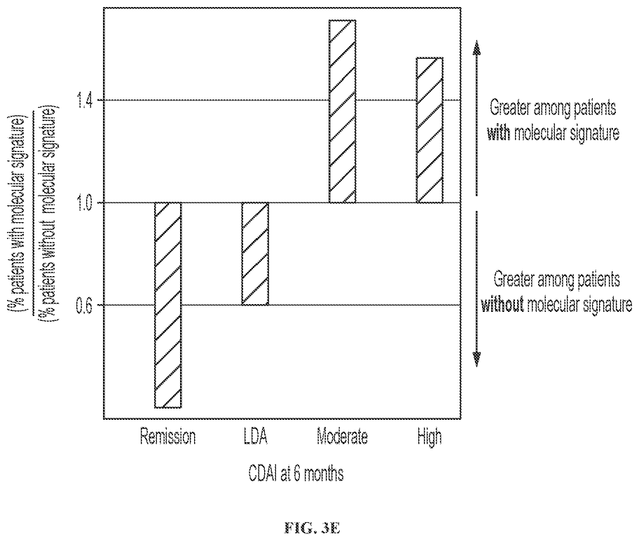

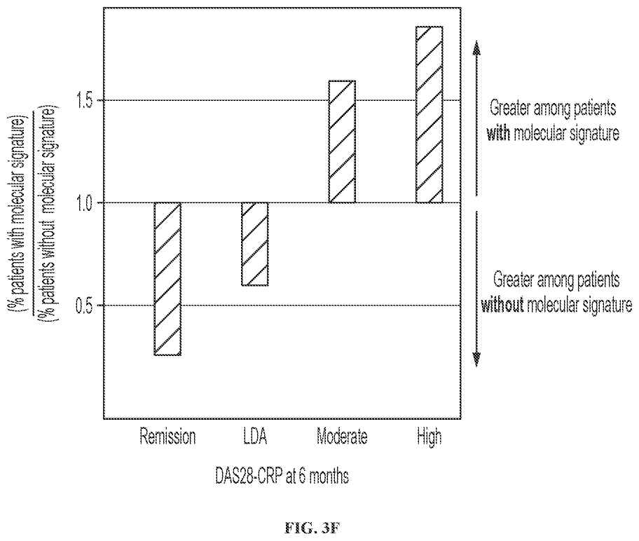

According to the primary endpoint of ACR50 response to TNFi therapy at 6 months, the MSRC stratified patients according to their likelihood of inadequate response to TNFi therapy with an AUC of 0.64 (FIG. 3A) and an odds ratio of 4.1 (95% CI: 2.0-8.3; p-value 0.0001) (Table 9).

Additional endpoints included assessment of ACR50 response at 3 months and response to treatment at 3 and 6 months according to ACR70, DAS28-CRP remission (<2.4) or LDA (<2.9) and CDAI remission (<2.8) or LDA (<10). The MSRC stratified patients according to their likelihood of inadequate response at both timepoints and response criteria with AUC values ranging from 0.59-0.74 (FIGS. 3A-3B) and significant odds ratios of 3.0-9.1 (p-value <0.01)(Table 7). Odds ratios describing whether patients with a molecular signature of non-response failed to achieve ACR70 or DAS28-CRP remission were significant at 6 months (p-value <0.0001), but not 3 months (p-values 0.07 and 0.34, respectively). Significant differences (p-value<0.002) in model scores between patients who did and did not have a molecular signature of non-response were observed for all response criteria except DAS28-CRP remission at 3 months (FIGS. 3B-3C). Furthermore, the fraction of patients who achieved remission and LDA at 6 months per CDAI and DAS28-CRP definitions was greater among those patients who did not have a molecular signature of non-response (FIGS. 3E-3F).

TABLE 9

| | |
|---|---|
| AUC | Odds ratio (95% CI; p-value) |

Prospective observational,
naïve

| ACR50, 3 months | 0.67 | 3.5 | (1.6-7.7; 0.002) |
|---|---|---|---|
| ACR50, 6 months | 0.64 | 4.1 | (2.0-8.3; 0.0001) |
| ACR70, 3 months | 0.67 | 2.6 | (1.0-6.7; 0.07) |
| ACR70, 6 months | 0.72 | 9.1 | (3.5-24.2; <0.0001) |
| CDAILDA, 3 months | 0.65 | 3.0 | (1.4-6.1; 0.005) |
| CDAILDA, 6 months | 0.68 | 3.6 | (1.8-7.2; 0.0002) |
| CDAI remission, 3 months | 0.70 | 3.4 | (1.3-8.7; 0.01) |
| CDAI remission, 6 months | 0.74 | 8.4 | (3.0-23.9; <0.0001) |
| DAS28-CRP LDA, 3 months | 0.69 | 3.3 | (1.5-7.1; 0.002) |
| DAS28-CRP LDA, 6 months | 0.67 | 3.4 | (1.7-6.8; 0.0007) |
| DAS28-CRP remission, 3 months | 0.59 | 1.5 | (0.7-3.2; 0.34) |
| DAS28-CRP remission, 6 months | 0.74 | 5.2 | (2.5-11.1; <0.0001) |
| Prospective observational, | | | |
| TNFi-exposed | | | |
| ACR50, 6 months | 0.67 | 3.3 | (1.5-7.4; <0.0001) |
| ACR70, 6 months | 0.76 | 7.3 | (2.3-23.3; 0.00004) |
| CDAILDA, 6 months | 0.74 | 7.5 | (3.2-17.6; <0.0001) |
| CDAI remission, 6 months | 0.84 | 25.4 | (3.2-200.6; <0.0001) |
| DAS28-CRP LDA, 6 months | 0.71 | 6.2 | (2.6-14.9; <0.0001) |
| DAS28-CRP remission, 6 months | 0.65 | 2.0 | (0.9-4.6; 0.1) |

Validation of the MSRC in a Prospective Observational Clinical Study Among TNFi-Exposed Patient Samples

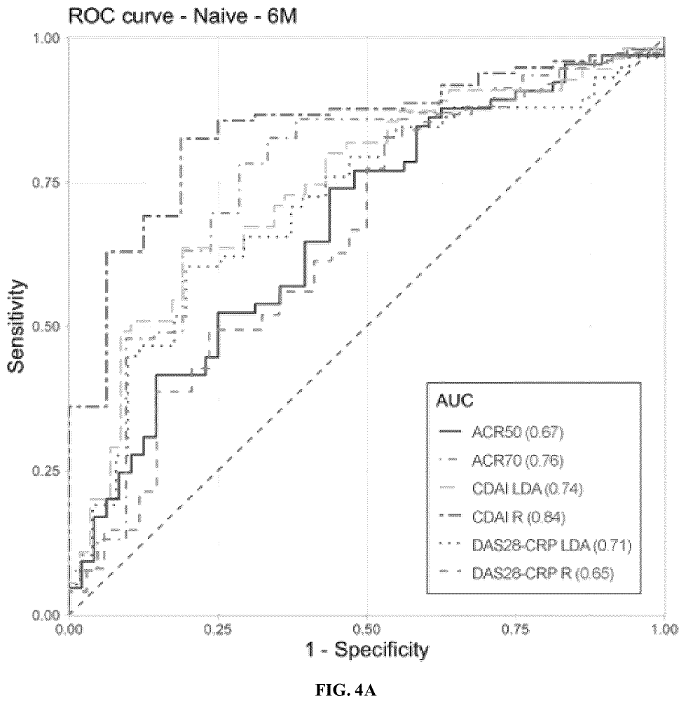

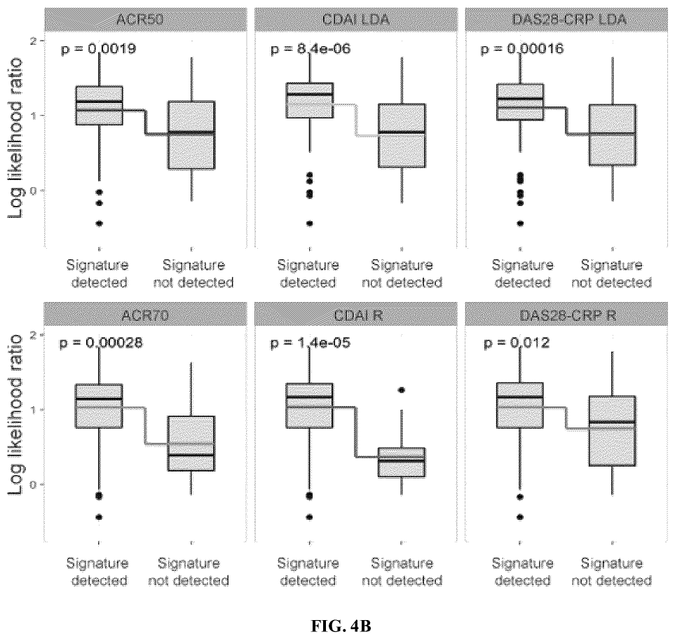

Among patients who completed the 24-week study, RNA blood samples at 3 months were available for 113 patients. Using the same MSRC as in the targeted therapy-naive analyses, 3-month patient samples were used to predict inadequate response to TNFi therapy. The molecular signature in these TNFi-exposed samples stratified patients according to inadequate response to treatment with AUC values of 0.65 to 0.84 (FIG. 4A). A molecular signature of non-response was detected for 40.7% (46/113) of TNFi-exposed patients and a significant difference in model scores (p-values <0.012) was observed between patients who did and did not have a molecular signature of non-response (FIG. 4B). This corresponded to significant odds ratios of 3.3-25.4 among patients with a molecular signature failing to have a response to treatment according to all criteria except for DAS28-CRP remission (Table 9).

### Discussion

Many targeted therapeutic options are available in RA, but therapy selection is a challenge because these options have similar treatment outcomes. Precision medicine tools are greatly needed to identify which patients have the appropriate disease biology for each targeted therapy. The blood-based MSRC described analyzes RNA sequencing data along with clinical features to accurately identify targeted-therapy naive and TNFi-exposed patients who were unlikely to have an adequate response to TNFi therapy. Among patients initiating their first targeted therapy, those with a molecular signature of non-response were three to nine times less likely to have an adequate response to a TNFi therapy (Table 7). When testing was performed after the patient had been receiving TNFi therapy for at least three months, patients with a molecular signature of non-response were as much as 25 times less likely to achieve remission. Furthermore, the molecular signature of non-response was predictive of inadequate response to TNFi therapy according to multiple clinically validated measures including ACR50, ACR70, DAS28-CRP and CDAI. The MSRC can inform provider decision-making at multiple occasions in the care pathway, such as before initial therapy selection or when targeted TNFi therapy does not result in treatment goals and a second therapy or dose escalation is being considered. By validating multiple response target definitions, the MSRC fits within multiple practice protocols, making it easy to understand, act upon and operationalize within clinical settings.

Precision medicine has improved patient outcomes in oncology and hematology by matching therapy selection to patients' unique biology. Yet even in these fields, predicting drug response, particularly from blood, has remained a challenging technical problem. In addition, machine learning and statistical approaches are prone to over-fitting to characteristics and attributes of the study cohort population. Unlike in oncology, the reliance on DNA analyses and biopsies of disease tissue is not readily amenable in RA patient care because synovial biopsies outside of clinical studies are rare and DNA sequence variations provide limited actionable information in RA. Studies of the AMPLE, AVERT, GO-BEFORE and GO-FORWARD trials have used baseline disease assessments such as DAS28, RAPID3, CDAI or SDAI to predict radiographic progression or magnetic resonance imaging-detected synovitis in response to treatment with a targeted therapy. Comparable AUC values were reported (0.54-0.72) but the odds ratios (1.01-1.65) were lower than those observed in this study (3.0-25.4). Additionally, the odds ratios in this study were consistent between the cross-validation CERTAIN cohort and the prospective NETWORK-004 cohort indicating that the MSRC is reproducible and generalizable across studies and patient populations. The technical challenges surrounding development of precision medicine tools underscores the importance of evaluating biomarkers relevant to disease biology and development of new approaches, such as network-based methods, as evidenced by the results of this study.

The network-based methods used in this study uncovered novel connections within disease biology. A survey of 248 US-based rheumatologists demonstrated that rheumatologists may welcome precision medicine advances in autoimmune diseases and may find value in this predictive drug response test. Selection of TNFi therapy declined by more than 80% (from 79.8% to as low as 11.3%) when rheumatologists were presented with a sample MSRC result indicating non-response. Furthermore, the majority of rheumatologists surveyed reported that test results can increase their confidence in prescribing decisions, improve medical decision-making and alter their treatment choices. Treatment selection guided by a precision medicine tool that predicts response to TNFi therapy was modeled to improve response rates to targeted therapies and result in healthcare cost savings.

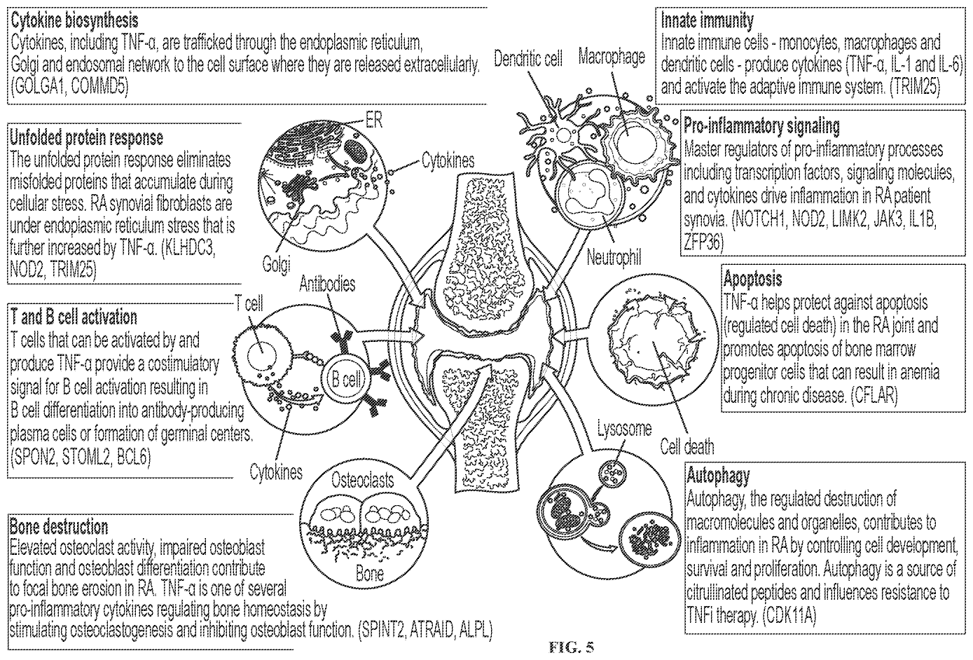

The RNA transcripts in the MSRC evaluate seemingly disparate aspects of disease biology that are nonetheless unified in the same network neighborhood on the human interactome and capture the diverse biology of RA and response to TNFi therapy. The proteins encoded by these transcripts influence biological processes including cellular homeostasis for adaptive and innate immune cells, production of TNF-α and other secreted signaling molecules, synovitis, and bone destruction (FIG. 5). TNF-α biology is robustly captured in the MSRC and features are involved in the production and release of TNF-α (e.g., COMMD5), and upstream or downstream TNF-α signaling events (e.g., NOTCH1). Identification of molecular characteristics expressed in circulating blood cells suggests that direct evaluation of joint physiology or biochemistry may not be essential to evaluating synovial phenotypes or response to treatment. The MSRC is rooted in RA disease biology and readily generalizes to the molecular phenotypes of an independent cohort of patients in the blinded study.

### Conclusions

Validation of the MSRC involved analysis of RNA sequencing data derived from blood samples of 391 RA patients who were treated with a TNFi therapy from two independent studies and patient populations, reproducing for the first time the predictive ability of molecular biomarkers using seemingly disparate RA biology. These findings demonstrate that direct evaluation of joint physiology or biochemistry may not be essential to predict response. Among patients who are naive to targeted therapy and those who are TNFi-exposed, patients with a molecular signature of non-response are unlikely to respond to TNFi therapy at 3 or 6 months as assessed by ACR50, ACR70, DAS28-CRP and CDAI. When providers use MSRC test results to stratify patients to treatment, patients who have a molecular signature of non-response to TNFi therapies can be directed to an alternative therapy to avoid expenses and potential toxicities without possible benefit. Those who lack this signature can proceed with TNFi therapy and possibly achieve an increased response rate relative to the unstratified population.

### Example 2—Clinical Longevity of an RNA Signature Panel for Prediction of Non-Response to Tumor Necrosis Factor Inhibitor Therapies in Rheumatoid Arthritis Patients

As a potentially debilitating autoimmune disease, rheumatoid arthritis (RA) is the telltale clinical presentation involving joint deterioration and chronic inflammation. Although no cure exists for the disease, RA patients do have a wide variety of therapies available that can mitigate symptoms and forestall joint destruction. Treatment guidelines indicate that early therapeutic intervention is important for delaying permanent loss of joint function accompanying structural damage to tissues. Once patients are diagnosed with RA, the first course of treatment applied can be a synthetic disease modifying antirheumatic drug (csDMARD), with methotrexate as an example first line option. RA patients whose symptoms are not sufficiently controlled with csDMARDs have a wide range of other therapies according to treatment guidelines, including targeted drugs for inhibiting interleukin-6 (IL-6), Janus kinase (JAK), and tumor necrosis factor-a (TNF). While targeted therapies indicated as the next step in treatment beyond csDMARDs, no one therapy is recommended over other targeted therapies in these circumstances and choice of therapy may be dependent upon non-clinical selection factors. This is demonstrated by the >80% of biologic-naive RA patients with symptoms insufficiently controlled by csDMARDs who are then directed toward anti-TNF therapies.

Within the large subset of patients that respond inadequately to csDMARDs and subsequently initiate anti-TNF medications, roughly 75-90% of these RA patients do not reach the intended therapeutic targets of low disease activity (LDA) or remission in guidelines from the American College of Rheumatology (ACR). Of the patients with RA just starting TNF inhibitors (TNFi), a 20% improvement in symptoms (ACR20) was seen in 50-70% of patients, a 50% ACR score improvement (ACR50) was observed in 30-40% of patients, and 15-25% of patients reached a 70% improvement (ACR70). No more than roughly 10-25% of biologic-naive RA patients who initiated TNFi treatment are reported as being able to achieve remission of RA symptoms, indicating that the broad application of TNFi therapy has an unmet need for precision medicine to reduce the likelihood of treatment cycling. Given the degenerative nature of RA over time, mitigating delays in reaching treatment targets can bring quality of life benefits to RA patients who are non-responders to anti-TNF treatments.

Under the present circumstances of TNFi use, previous studies indicate that RA patients who initiate such treatment are receiving a therapeutic regimen thatis sub-optimal for their specific biology. This results in a substantial amount of monetary waste within the healthcare system in paying for TNFi therapy in patients who will not benefit from the treatment, and this also delays those RA patients from being directed toward therapies with an alternative mechanism of action that may be better suited to their specific circumstances. Given this current scenario is unhelpful for most RA patients and healthcare resource efficiency alike, the introduction of a validated biomarker panel with the capacity to predict patient non-responsiveness to TNFi can be highly beneficial and well-received by rheumatologists. A proprietary biomarker panel and predictive algorithm analyzing 19 RNA transcripts, laboratory tests for anti-cyclic citrullinated peptide (anti-CCP), and 3 clinical metrics (BMI, gender, and patient global assessment) developed by Scipher Medicine, known as PrismRA, has been demonstrated to be predictive of biologic-naive RA patient response to anti-TNF therapies. At present, the length of time for which PrismRA results remain valid after patients begin TNFi treatment is unknown. This current study was crafted to evaluate the stability of PrismRA predictions throughout a time course of TNFi therapy with the intention of determining the long-term clinical meaningfulness of PrismRA scores on both the population and individual levels.

Study Populations

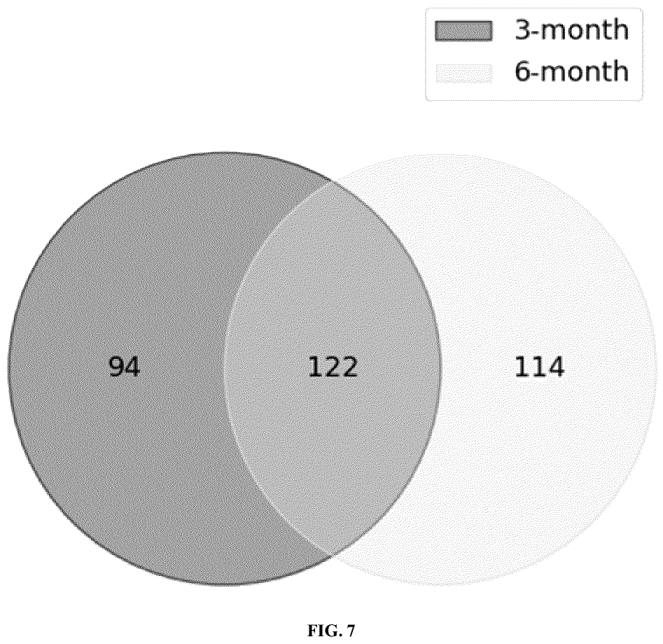

The demographics of the patient population assessed herein are outlined in Table 10. A total of 452 whole-blood samples and accompanying clinical measurements were obtained from 330 patients with rheumatoid arthritis. Samples were collected from the RA patients after the patients had initiated anti-TNF therapy. All patients included in the study were naive to RA biologics prior to starting on a TNFi course of treatment. RA patient blood samples were collected at 3 months or 6 months following the start of TNFi initiation, with a cross-section of patients who provided samples at both time points. Within the patient populace, 94 patients provided samples at 3-months post TNFi initiation only, 114 patients provided samples at the 6-month time point only, and 122 provided samples at both the 3-month and 6-month time points. Overlap amongst the three patient groups and two sample collection time points is shown in FIG. 7. All patients involved in this study provided informed consent, with approvals from an institutional review board obtained before any sample collection or study participation by patients took place. Selection of TNFi therapies and associated dosages were at the rheumatologists' discretion for all patients.

Clinical Evaluation and Response to Anti-TNF Therapy

Clinical response to anti-TNF therapies were assessed at baseline, 3-month, and 6-month visits according to criteria defined for ACR, Clinical Disease Activity Index (CDAI), and Disease Activity Score 28 with C-reactive protein (DAS28-CRP). The ACR measurements of ACR50, and ACR70 were defined as when an individual demonstrated ≥50%, or ≥70% improvement in the 28 tender joint count, the 28 swollen joint count, and in a minimum of three of the five clinical values used in evaluating an RA patient's disease state. Whole blood samples in PAXgene RNA blood tubes were collected at each visit. Rheumatoid factor (RF) and anti-cyclic citrullinated protein (anti-CCP) antibody serostatus measurements were established at patients' baseline sampling points. The variables used in evaluating an RA patient's disease state included the Health Assessment Questionnaire disability index, patient global assessment, provider global assessment, CRP and anti-CCP levels, and patient-reported pain. Clinical assessment data were also used to determine if patients met the clinical thresholds for CDAI low disease activity (CDAI-LDA), CDAI remission (CDAI-R), DAS28-CRP low disease activity (DAS28-CRP-LDA), and DAS28-CRP remission (DAS28-CRP-R).

RNA Isolation, Preparation, and Sequencing Analysis

PAX-gene Blood RNA Tubes were used to collect blood samples for total RNA isolation. MagMax™ for Stabilized Blood PAXgene Tubes RNA Isolation Kit from Thermo Fisher Scientific was used for RNA sample preparation according to protocols from the manufacturer. RNA within a mass range of 100-1000 ng were processed using a KAPA RNA HyperPrep Kit with RiboErase (HMR) Globin. An Agilent Bioanalyzer automated electrophoresis platform was used to evaluate the quality of collected RNA, while a NanoDrop ND-8000 spectrophotometer was used for RNA quantification. RNA samples were sequenced using an Illumina NovaSeq 6000 platform with a Clinical Laboratory Improvement Amendments (CLIA)-validated diagnostic assay. Gene expression across entire genomes was determined from processed sequence data. For inclusion in sample analysis, RNA samples were required to have a TapeStation RIN>4, an RNA concentration >10 ng/μL, a sequencing library yield >10 nM, a perfect base-pair index percentage >85, a percentage of bases over Phred score 30>75, a mean quality Phred score >30, a median Phred score >25, and a lower quartile Phred score >10 for all bases in the RNA molecules.

TNFi Response Predictive Model

A TNFi therapy response classification model was trained using 245 samples collected from RA patients using panel of 23 selected biomarkers. Model building was done using MLPClassifier package available in Python's machine learning library sklearn.

Statistical Analysis

Performance of the PrismRA biomarker panel was evaluated using the area under (AUC) receiver operating characteristic (ROC) curves. MSRS model cutoff used for odds ratio calculation was selected based on previous validation results. Odds ratio was calculated. Python 3.7.6 was used to perform all statistical analyses and data processing procedures. All values classifiable as continuous data were represented with mean, standard deviation, median, minimum, maximum, and observation count as appropriate. For categorical variables, values were summarized using frequency counts and percentages. For determination of confidence intervals (CI), continuous data CI were obtained using a t distribution, while CI for categorical variables were determined using an exact method. Two-sided tests were applied in all circumstances at the 0.05 significance level unless otherwise stated.

PrismRA Maintains Performance Throughout Anti-TNF Exposure Time Course

Table 10 shows demographics of the patient population evaluated in this study. In total, 452 samples collected from 330 RA patients who had recently initiated TNF therapy were evaluated. Samples were collected at 3-months after TNF initiation or at 6-months after TNF initiation. FIG. 7 shows the overlap of patients who provided samples at the 3-month and 6-month time points. Out of the 330 patients in the study, 94 provided samples at only the 3-month time point, 122 provided samples at both the 3-month and 6-month time points, and 114 provided samples at only the 6-month time point.

TABLE 10

| Characteristic | n = 330 |
|---|---|
| age, mean (SD) | 55.5 (12.5) |
| gender, female/male | 238/92 |
| CCP, positive/negative/unknown | 211/104/15 |
| RF, positive/negative/unknown | 233/83/14 |
| Methotrexate, yes/no | 235/95 |
| Prednisone, yes/no | 75/255 |
| Plaquenil, yes/no | 50/280 |
| Azulfidine, yes/no | 10/320 |
| Arava, yes/no | 22/308 |
| TNF, | 124/107/65/9/25 |
| Humira ®/Enbrel ®/Remicade ®/Simponi ®/ | |
| Cimzia ® | |

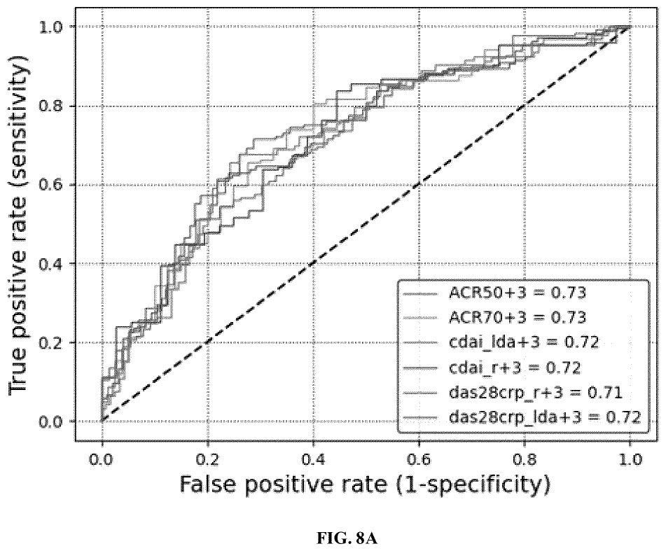

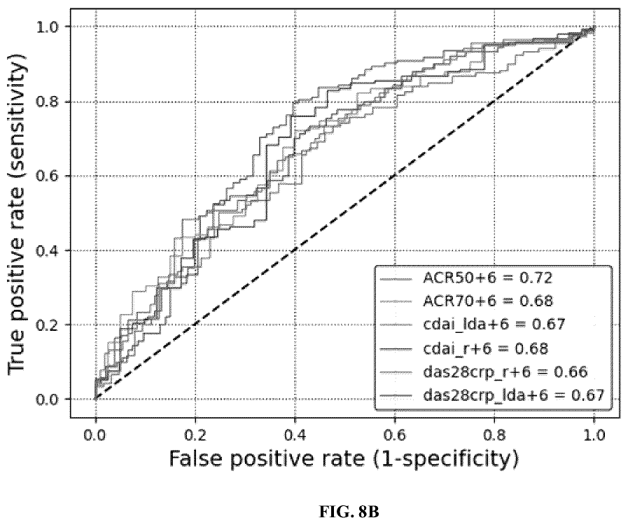

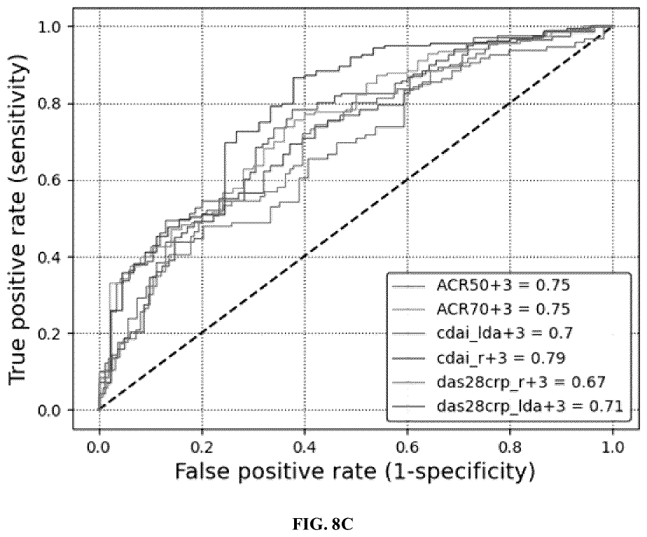

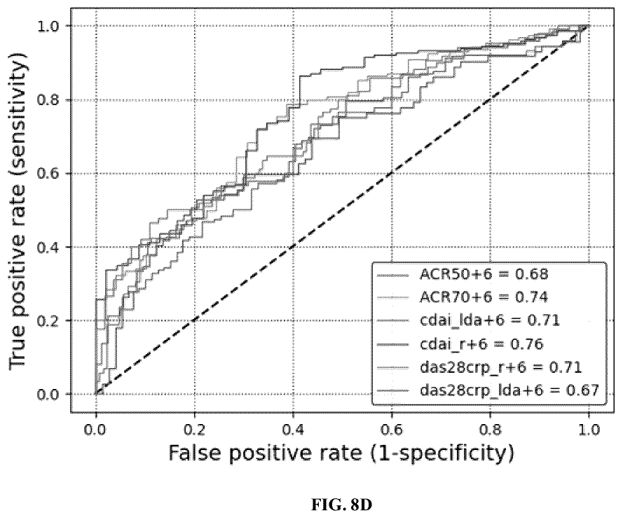

A molecular signature response classifier (MSRC) was used to predict therapeutic response to TNF therapy using patient data collected at the two timepoints. Patient response to TNF therapy was assessed at +3 months and +6 months after the time of sample collection using seven different clinically accepted response definitions (ACR20, ACR50, ACR70, CDAI-R, CDAI-LDA, DAS28-CRP-R, and DAS28-CRP-LDA). See materials and methods for more details. FIGS. 8A-8D show ROC curves which were generated by comparing the MSRC scores to the +3 month and +6 month therapeutic outcomes.

Comparable performance was observed when using 3-month data (FIGS. 8A, 8C) as compared to using 6-month data (FIGS. 8B, 8D) to make the prediction. Across the various response definitions, AUC's ranged from 0.66-0.73 when using 3-month data and 0.67-0.75 when using 6-month data. Similar performance was observed when comparing model predictions to therapeutic response outcomes at +3 months (FIGS. 8A, 8C) and +6 months (FIGS. 8B, 8D) after the time of data collection, with AUC's ranging from 0.67-0.79 and 0.66-0.76, respectively. Table 11 provides a summary of the odds ratios observed among each of the samples and endpoint definitions. For all models evaluated, statistically significant differences in score distribution between responders and nonresponders was observed (p<0.001).

TABLE 11

| Time point | Endpoint | AUC | OR | [CI] |
|---|---|---|---|---|
| 3-month | ACR50 + 3 | 0.73 | 4.83 | [2.42-9.62] |
| 3-month | das28crp_r + 3 | 0.71 | 4.65 | [2.36-9.15] |
| 3-month | das28crp_lda + 6 | 0.67 | 3.53 | [1.87-6.64] |
| 3-month | das28crp_lda + 3 | 0.72 | 5.73 | [2.99-10.99] |
| 3-month | cdai_r + 6 | 0.68 | 3.12 | [1.35-7.18] |
| 3-month | cdai_r + 3 | 0.72 | 5.03 | [1.86-13.54] |
| 3-month | das28crp_r + 6 | 0.66 | 4.42 | [2.1-9.29] |
| 3-month | cdai_lda + 3 | 0.72 | 5.49 | [2.98-10.11] |
| 3-month | cdai_lda + 6 | 0.67 | 3.04 | [1.67-5.53] |
| 3-month | ACR70 + 6 | 0.68 | 3.65 | [1.52-8.76] |
| 3-month | ACR70 + 3 | 0.73 | 4.63 | [1.85-11.61] |
| 3-month | ACR50 + 6 | 0.72 | 3.63 | [1.88-6.99] |
| 6-month | cdai_lda + 3 | 0.7 | 4.32 | [2.21-8.43] |
| 6-month | cdai_lda + 6 | 0.71 | 5.98 | [2.91-12.26] |
| 6-month | ACR70 + 3 | 0.75 | 7.71 | [2.64-22.48] |
| 6-month | cdai_r + 3 | 0.79 | 7.19 | [2.46-21.0] |
| 6-month | cdai_r + 6 | 0.76 | 7.14 | [2.45-20.87] |
| 6-month | das28crp_lda + 3 | 0.71 | 4.69 | [2.16-10.18] |
| 6-month | ACR50 + 6 | 0.68 | 3.34 | [1.69-6.59] |
| 6-month | das28crp_lda + 6 | 0.67 | 4.8 | [2.07-11.13] |
| 6-month | das28crp_r + 3 | 0.67 | 5.22 | [2.14-12.72] |
| 6-month | ACR50 + 3 | 0.75 | 6.36 | [2.92-13.87] |
| 6-month | ACR70 + 6 | 0.74 | 5.18 | [2.08-12.88] |
| 6-month | das28crp_r + 6 | 0.71 | 8.5 | [2.82-25.58] |

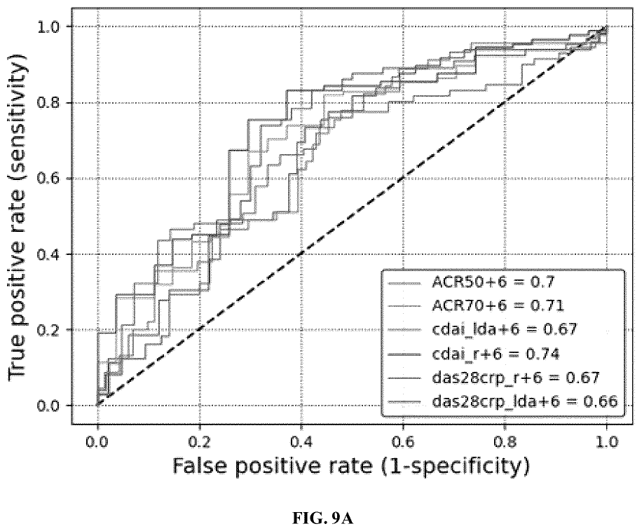

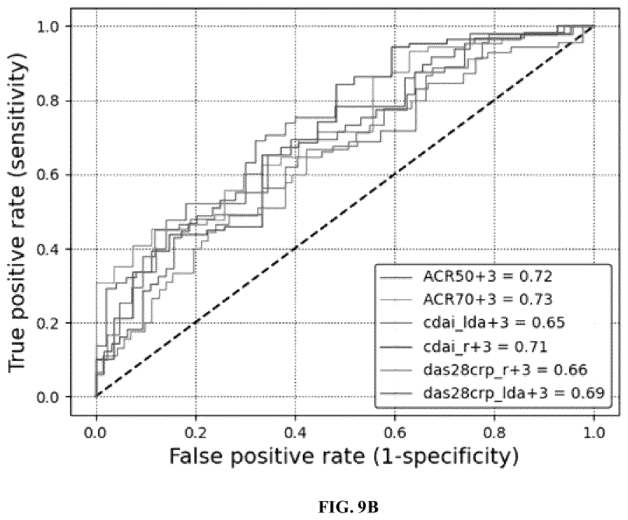

Stability of MSRC predictions were further evaluated by comparing model performance among the 122 patients who provided both 3-month and 6-month samples (FIGS. 9A-9B). In order to ensure outcomes were consistent, response was evaluated at +9 months after TNF therapy was initiated (+6 months from the 3-month sample collection timepoint and +3 months from the 6-month sample collection timepoint). Across the various response definitions, AUC's ranged from 0.66-0.74 when using data collected at 3 months after TNF initiation and 0.65-0.73 when using data collected at 6 months after TNF initiation. In both cases, statistically significant differences in MSRC score distribution between responders and nonresponders was observed (p<0.001).

PrismRA Predictions Demonstrate Stability Throughout Anti-TNF Exposure Timecourse

In order to assess the longitudinal stability of PrismRA response predictions on an individual basis, we first investigated the stability of the response outcome labels among the 122 patients for which 3-month and 6-month data was available. Table 12 details the degree of agreement between outcomes when considering data collected at 3 months and 6 months after TNF initiation. On average, +3 month outcomes were consistent 73.900 of the time across the various endpoint definitions while +6 month outcomes were consistent 80.9% of the time. Among the patients who were not consistent, similar proportions changed from nonresponders to responders as nonresponders to responders. For +3 month outcomes, on average 14% change from nonresponder to responder and 12% changed from responder to nonresponder. For +6 month outcomes, 8.5% changed from nonresponder to responder and 10.5% changed from responder to nonresponder.

TABLE 12

| Endpoint | NR-NR | R-R | agreement | NR-R | R-NR | disagreement |
|---|---|---|---|---|---|---|
| ACR50 + 3 | 52 (44.1%) | 33 (28.0%) | 72.0% | 18 (15.3%) | 15 (12.7%) | 28.0% |
| ACR50 + 6 | 55 (49.1%) | 37 (33.0%) | 82.1% | 8 (7.1%) | 12 (10.7%) | 17.9% |
| ACR70 + 3 | 79 (66.9%) | 17 (14.4%) | 81.4% | 11 (9.3%) | 11 (9.3%) | 18.6% |
| ACR70 + 6 | 77 (68.1%) | 20 (17.7%) | 85.8% | 8 (7.1%) | 8 (7.1%) | 14.2% |
| cdai_lda + 3 | 27 (22.7%) | 55 (46.2%) | 68.9% | 19 (16.0%) | 18 (15.1%) | 31.1% |
| cdai_lda + 6 | 31 (27.2%) | 59 (51.8%) | 78.9% | 11 (9.6%) | 13 (11.4%) | 21.1% |
| cdai_r + 3 | 85 (71.4%) | 17 (14.3%) | 85.7% | 11 (9.2%) | 6 (5.0%) | 14.3% |
| cdai_r + 6 | 80 (70.2%) | 19 (16.7%) | 86.8% | 6 (5.3%) | 9 (7.9%) | 13.2% |
| das28crp_r + 3 | 54 (47.4%) | 29 (25.4%) | 72.8% | 15 (13.2%) | 16 (14.0%) | 27.2% |
| das28crp_r + 6 | 53 (50.0%) | 31 (29.2%) | 79.2% | 11 (10.4%) | 11 (10.4%) | 20.8% |
| das28crp_lda + 3 | 33 (28.9%) | 43 (37.7%) | 66.7% | 23 (20.2%) | 15 (3.2%) | 33.3% |
| das28crp_lda + 6 | 34 (32.1%) | 46 (43.4%) | 75.5% | 12 (11.3%) | 14 (13.2%) | 24.5% |

Stability of MSRC predictions throughout the TNF therapy time course was evaluated by comparing predictions made using data collected three months after TNF therapy initiation to those made using data collected six months after TNF therapy initiation. Out of the 122 patients for which both 3-month and 6-month data was available, 97 (81.5%) had consistent class predictions between the two time points, while 22 (18.5%) had different predictions between the two time points. Among the 18.5% that changed, 9 changed from nonresponder to responder and 12 changed from responder to nonresponder.

### Discussion

Biologic therapies for rheumatoid arthritis can be aimed at a range of different targets (TNF, IL-6, and JAK) and have roughly equivalent benefits when patients respond to the therapies, yet the most frequent choice of biologic treatment by rheumatologists is a TNFi. Without the presence of additional clinical guidance, the predominant use of TNFi is sure to continue, which means that the 90% of patients not responding sufficiently to csDMARDs will be put on a medication that has a 70% chance of failing to meet treat-to-target thresholds for RA. However, these negative outcomes can be mitigated through implementation of a clinical panel that can determine if a patient will respond to TNFi therapy. The PrismRA predictive biomarker panel has been clinically validated in previous studies to successfully classify RA patients as TNFi responders or non-responders.

One goal of this study was to determine the efficacy of the PrismRA MSRC throughout a timecourse of TNF therapy. On a population level, the model performance is good and reflects previous validation results. The MSRC was able to distinguish nonresponders from responders with AUCs ranging from 0.61-0.69 when using 3-month data and 0.64-0.73 when 6-month data. This corresponded well with observations by Mellors et al., where the biomarker response panel successfully identified TNFi non-responders with an odds ratio of 6.57. Additionally, prior validation of the MSRC by Cohen et al. successfully stratified a patient's likelihood of being a TNFi non-responder with an odds ratio of 4.1 according to an ACR50 response end point at 6 months.

The predictive performance for the +3-month and +6-month outcomes remained consistent regardless of when samples were collected throughout an anti-TNF therapy timeline. Stability of PrismRA outcomes indicate that the biomarker panel may be employed at any time during a TNFi treatment course while still offering an effective prediction about how a patient will respond to the therapy in another 3 to 6 months. Rather than offering valid treatment guidance merely at the beginning of treatment and changing over time, this study shows the long-term validity of this biomarker panel during a TNFi therapy time course.

Outcomes from this study do have a certain degree of variability, which can be inferred from the consistency rates of roughly 74% for +3-month outcomes and 81% for +6-month outcomes. The occurrence of incorrect predictions or patients switching between responder and non-responder in consecutive measurements both highlight the importance to understand the natural outcome variability for properly characterizing model performance.

Among outcomes that disagree, roughly the same proportion of patients will change from responder to non-responder as will change from non-responder to responder. On average, the proportion of non-responders changing to responders was 14% at the +3-month mark and 8.5% at the +6-month timepoint, while responders were found to switch to non-responders 12% of the time at +3-months and 10.5% at +6-months. Since there is a small chance that a patient on anti-TNF therapy who tests as a non-responder may flip status to become a responder, some clinicians may be inclined to keep non-responding patients on a TNFi in the event that they eventually respond. However, the time-sensitive and debilitating nature of RA disease progression can warrant a clinical decision with a higher probability of success than waiting out an ineffective therapy for the chance of eventual response. In their 2021 guidelines, the American College of Rheumatology adjusted the recommendation for bDMARDs like anti-TNF therapies, where patients who are not at target improvement should be switched to a bDMARD of a different drug class rather than a different bDMARD in the same class. Among the alternatives to TNF inhibitors, options such as inhibitors of IL-6, IL-6 receptors, and JAK have all been reported to be roughly as effective as TNF inhibitors (29-36). Moreover, the other classes of bDMARDs have been reported to remain effective in RA patients even after use of and non-response to anti-TNF therapies, so patients who may switch from responder to non-responder after an initial TNFi treatment still have biologics as an effective therapeutic option (37-39). The updated ACR recommendations and availability of effective alternative to TNFi therapies substantiate the motivation for stratifying patients as responders or non-responders to TNFi treatment so that no time is wasted either trialing a therapy with a poor population response rate or continuing on a medication to which a patient no longer responds. In a study regarding perceived clinical utility of the PrismRA panel, Pappas et al. found that an MSRC capable of classifying patients' TNFi response may be well received by rheumatologists. Of the 248 clinicians surveyed, 92% felt that the test results can raise their confidence when deciding on RA patients' treatment, and roughly 80% of the surveyed rheumatologists agreed that this type of biomarker panel can improve medical decision-making.

The MSRC showed a high level of consistency when comparing predictions made using 3-month data as compared to the 6-month data. Results compared between measurements taken at these two time points remained consistent >810% of all instances. These data suggest that even if the MSRC is given at various points through a TNFi time course, the panel will still yield consistent results. With results establishing the validity and utility of using an MSRC to identify non-responders to TNFi treatment at the point of therapy selection (24,26,40-43), this longitudinal study shows that physicians may or may not retest their patients after the initial test. Correspondingly, patients are recommended to receive the PrismRA test when a prescription change is warranted secondary to disease progression or side effects from current therapy. Further studies measuring the PrismRA testing interval are planned to be conducted to better define the patient testing recommendations.

Knowing that there is sustained accuracy of the PrismRA test, further waste and inefficiencies indicative of repeat testing or indeterminate results can be avoided and the cost savings of placing patients on the most effective treatment based on their molecular profile can be realized. An approximation of the cost savings from predicting non-response to TNF inhibitors by PrismRA in clinical decision-making was described by Bergman et al. When modeling standard-of-care biologic pharmacy treatment costs compared to using PrismRA stratification in 12 months of RA patient treatment, a 22% decrease in costs spent on ineffective treatments and 5% cost of RA treatment overall can be obtained. Among the Medicare-eligible population, these savings were equated to an annual per-patient $6668 reduction in spending on ineffective treatments. When considering that PrismRA stratification causes both direct reduction in ineffective treatments as well as bringing indirect value by mitigating disease progression, the multifold benefits possible in RA reinforce the value of advancing precision medicine in the clinical space.

The foregoing has been a description of certain non-limiting embodiments of the subject matter described within. Accordingly, it is to be understood that the embodiments described in this specification are merely illustrative of the subject matter reported within. Reference to details of the illustrated embodiments is not intended to limit the scope of the claims, which themselves recite those features regarded as essential.

It is contemplated that systems and methods of the claimed subject matter encompass variations and adaptations developed using information from the embodiments described within. Adaptation, modification, or both, of the systems and methods described within may be performed by those of ordinary skill in the relevant art.

Throughout the description, where systems are described as having, including, or comprising specific components, or where methods are described as having, including, or comprising specific steps, it is contemplated that, additionally, there are systems encompassed by the present subject matter that consist essentially of, or consist of, the recited components, and that there are methods encompassed by the present subject matter that consist essentially of, or consist of, the recited processing steps.

It should be understood that the order of steps or order for performing certain action is immaterial so long as any embodiment of the subject matter described within remains operable. Moreover, two or more steps or actions may be conducted simultaneously.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-20. (canceled)

21. A method of treating a subject suffering from rheumatoid arthritis, the method comprising:
administering to the subject an anti-TNF therapy,
wherein the subject has been predicted to be responsive to the anti-TNF therapy based at least in part on a trained machine learning classifier that distinguishes between responsive subjects and non-responsive subjects who have received the anti-TNF therapy,
wherein the trained machine learning classifier distinguishes between the responsive subjects and the non-responsive subjects, based at least in part on analyzing an expression level in the subject of a set of genes.

22. The method of claim 21, wherein the trained machine learning classifier further analyzes:
presence of one or more single nucleotide polymorphisms (SNPs) in a sequence of one or more genes that are expressed in the subject; or
presence of one or more clinical characteristics of the subject.

23. The method of claim 22, wherein the one or more clinical characteristics of the subject comprise a member selected from the group consisting of body-mass index (BMI), gender, age, race, previous anti-TNF therapy treatment, disease duration of rheumatoid arthritis, C-reactive protein level, presence of anti-cyclic citrullinated peptide, presence of rheumatoid factor, patient global assessment, and treatment response rate to anti-TNF therapy.

24. The method of claim 21, wherein the anti-TNF therapy comprises infliximab, adalimumab, etanercept, certolizumab pegol, golimumab, or a biosimilar thereof.

25. The method of claim 21, wherein the anti-TNF therapy comprises adalimumab, infliximab, etanercept, or a biosimilar thereof.

26. The method of claim 21, wherein the trained machine learning classifier predicts the subject to be responsive to the anti-TNF therapy using a non-linear relationship between (i) an expression level of one or more genes identified in the subject and (ii) responsiveness or non-responsiveness to the anti-TNF therapy.

27. The method of claim 21, wherein the trained machine learning classifier is trained using expression levels of a set of genes in (i) a first set of subjects with rheumatoid arthritis who were responsive to the anti-TNF therapy and (ii) a second set of subjects with rheumatoid arthritis who were non-responsive to the anti-TNF therapy.

28. The method of claim 21, wherein the trained machine learning classifier comprises a neural network or a random forest.

29. The method of claim 21, wherein the trained machine learning classifier predicts that subjects within a population are responsive to the anti-TNF therapy with a true negative rate (TNR) of at least about 60%.

30. The method of claim 21, wherein the set of genes comprises ALPL, ATRAID, BCL6, CDK11A, CFLAR, COMMD5, GOLGA1, IL1B, IMPDH2, JAK3, KLHDC3, LIMK2, NOD2, NOTCH1, SPINT2, SPON2, STOML2, TRIM25, or ZFP36.

31. The method of claim 30, wherein the set of genes comprises ALPL, BCL6, CDK11A, CFLAR, IL1B, JAK3, LIMK2, NOD2, NOTCH1, TRIM25, or ZFP36.

32. The method of claim 21, wherein the trained machine learning classifier predicts that subjects within a population are responsive to the anti-TNF therapy with a negative predictive value (NPV) of at least about 85%.

33. The method of claim 21, wherein the trained machine learning classifier predicts that subjects within a population are responsive to the anti-TNF therapy with an area under the curve (AUC) of at least about 70%.

34. The method of claim 21, wherein the trained machine learning classifier predicts that subjects within a population are responsive to the anti-TNF therapy with an accuracy of at least about 90%.

35. The method of claim 22, wherein the one or more SNPs comprise a member selected from the group consisting of chr1.161644258, chr1.2523811, chr11.107967350, chr17.38031857, chr7.128580042, rs10774624, rs10985070, rs11889341, rs1571878, rs1633360, rs17668708, rs1877030, rs1893592, rs1980422, rs2228145, rs2233424, rs2236668, rs2301888, rs2476601, rs3087243, rs3218251, rs331463, rs34536443, rs34695944, rs4239702, rs4272, rs45475795, rs508970, rs5987194, rs657075, rs6715284, rs706778, rs72634030, rs73013527, rs73194058, rs773125, rs7752903, rs8083786, and rs9653442.
